(19) United States
(12) Patent Application Publication
(10) Pub. No.: US 2002/0132223 A1
(43) Pub. Date: Sep. 19, 2002

(54) Methods for modulating activity of the FXR nuclear receptor

(75) Inventors: Barry M. Forman, Newport Beach, CA (US); Haibo Wang, Fresno, CA (US)

Correspondence Address: ROTHWELL, FIGG, ERNST & MANBECK, P.C., 1425 K STREET, N.W., SUITE 800, WASHINGTON, DC, 20005, US

(73) Assignee: City of Hope, Duarte, CA (US)

(21) Appl. No.: 09/971,067

(22) Filed: Oct. 5, 2001

Related U.S. Application Data
(63) Continuation-in-part of application No. 09/533,862, filed on Mar. 24, 2000.
(60) Provisional application No. 60/126,334, filed on Mar. 26, 1999.

(51) Int. Cl. C12Q 1/00; A61K 31/496

(52) U.S. Cl. 435/4; 514/254.05; 514/374; 514/378; 514/381; 514/365; 514/372; 514/406

(57) ABSTRACT

The present invention relates to methods and compositions for modulating genes which are controlled by the FXR nuclear hormone receptor such as Cyp7a, Cyp8b, phospholipid transfer protein, ileal bile acid binding protein, sodium taurocholate cotransporter protein, liver fatty acid binding protein and bile salt export pump. In a preferred embodiment, the method involves modulation of the gene encoding Cyp7a, the enzyme responsible for a major pathway in the elimination of cholesterol. The invention also relates to methods for screening compounds which bind to and activate or inhibit the FXR nuclear hormone receptor and compounds which activate or inhibit the FXR nuclear hormone receptor.

### CROSS REFERENCE TO RELATED APPLICATIONS

This application is a continuation-in-part of prior co-pending application Ser. No. 09/533,862, filed Mar. 24, 2000, which claims priority from provisional application Serial No. 60/126,334, filed Mar. 26, 1999.

### BACKGROUND

1. Technical Field

This invention relates to methods of modulating physiological processes dependent on the FXR “orphan receptor” and to methods for identifying compounds which modulate such processes. Compounds for modulating FXR and the processes dependent on FXR also are provided.

2. Discussion of the Background Art

Cholesterol is essential for a variety of cellular activities, including membrane biogenesis, steroid and bile acid biosynthesis, caveolae formation and covalent protein modification. The widespread utilization of cholesterol in different metabolic pathways indicates that minimal blood concentrations must be maintained for optimal health. On the other hand, an excess of circulating cholesterol is a major risk factor in the development of atherosclerotic heart disease, the single largest cause of mortality in the United States which accounts for nearly 500,000 deaths each year.

Circulating cholesterol levels are regulated by cellular uptake, synthesis and degradation (Brown and Goldstein, Cell, 89:331-340, 1997). Removal of excess cholesterol from the body is complicated by the fact that it is an insoluble lipid, most of which is embedded within cell membranes. The major route for cholesterol degradation is metabolic conversion to bile acids, which are less hydrophobic and hence more easily removed from the cell than cholesterol. The conversion to bile acids occurs exclusively in the liver. One chemical pathway for this conversion is initiated by cholesterol 7α-hydroxylase (Cyp7a), the rate-limiting enzyme in this pathway. In humans, cholesterol is converted to bile acids by both the 7α-hydroxylase and, the sterol 27-hydroxylase pathways.

In vivo, bile acids are metabolized by hepatocytes and intestinal microorganisms, producing a large number of different products. The existence of so many chemically related products and complex biochemical pathways make the isolation and study of the effects of individual components difficult, particularly in the whole animal. See Elliott and Hyde, Am. J. Med., 51:568-579 (1971). For example, chenodeoxycholic acid (CDCA; 5β-cholanic acid-3α, 7α-diol) and cholic acid (CA; 5β-cholanic acid-3α, 7α, 12α-triol) are two of the major end products of bile acid biosynthesis (Chiang, Front. Biosci., 3:D176-193 (1998); Vlahcevic et al., Hepatology, 13:590-600 (1991)). Both are produced exclusively in the liver, where they can be further metabolized by conjugation with taurine or glycine. These bile acids are secreted into the intestine, reabsorbed in the ileum, and transported back to the liver via the portal circulation. During their transit in the intestine, primary bile acids such as CDCA and CA undergo microbial mediated 7α-dehydroxylation and are converted to lithocholic acid (LCA; 5β-cholanic acid-3α-ol) and deoxycholic acid (DCA; 5β-cholanic acid-3α, 12α-diol), respectively (Elliott and Hyde, Am. J. Med., 51:568-579 (1971); Hylemon, “Metabolism of Bile Acids in Intestinal Microflora” in Sterols and Bile Acids, H. Danielsson and J. Sjovall, eds. (New York, Elsevier), pp. 331-344, 1985).

In addition to their metabolic functions, bile acids also act as signaling molecules that negatively regulate their own biosynthesis. In particular, biliary components act in a negative feedback loop that limits bile acid production by inhibiting expression of the Cyp7a enzyme. While it is known that several bile acid components can induce this negative feedback regulation, the nature of the bile acid sensor which transduces the bile acid signal and the mechanism by which it does so have heretofore remained unknown. Inhibition of Cyp7a is known to occur at the transcriptional level, however, and negative bile acid response elements have been found in the Cyp7a promoter. Bile acids also have been shown to down-regulate sterol 27-hydroxylase, the enzyme involved in conversion of cholesterol to bile acids through a different pathway. See Twisk et al., Biochem. J., 305:505-511 (1995).

Nuclear receptors are ligand-modulated transcription factors that mediate the transcriptional effects of steroid, thyroid and retinoid hormones. These receptors have conserved DNA-binding domains (DBD) which specifically bind to the DNA at cis-acting elements in the promoters of their target genes and ligand binding domains (LBD) which allow for specific activation of the receptor by a particular hormone or other factor. Transcriptional activation of the target gene for a nuclear receptor occurs when the circulating ligand binds to the LBD and induces a conformation change in the receptor that facilitates recruitment of a coactivator. Coactivator recruitment results in a receptor complex which has a high affinity for a specific DNA region and which can modulate the transcription of the specific gene. Recruitment of a coactivator after agonist binding allows the receptor to activate transcription. Binding of a receptor antagonist induces a different conformational change in the receptor such that coactivator recruitment results in non-productive interaction with the basal transcriptional machinery of the target gene. As will be apparent to those skilled in the art, an agonist of a receptor that effects negative transcriptional control over a particular gene will actually decrease expression of the gene. Conversely, an antagonist of such a receptor will increase expression of the gene.

At least two classes of nuclear receptor coactivators have been identified. The first class includes the CBP and SRC-1 related proteins that modulate chromatin structure by virtue of their histone acetylase activity. A second class includes PBP/DRIP 205/TRAP 220 which is part of a large transcriptional complex that is postulated to interact directly with the basic transcriptional machinery.

In addition to the known classical nuclear hormone receptors that respond to specific, identified hormones, several orphan receptors have been identified which lack known ligands. These orphan receptors include, for example, FXR, CARβ, PPARα, PPARδ, TR2-11, LXRα, GCNF, SF1, RORα, Nurr1, DAX and ERR2. The orphan receptor FXR (farnesoid X-activated receptor) is known to inhibit Cyp7a. It binds to its response element as a heterodimer with RXR (9-cis retinoic acid receptor) which can be activated by RXR ligands.

It has been hypothesized that orphan receptors act as sensors for some metabolic signals, including fatty acids, prostanoids and metabolites of farnesol and cholesterol. Ever since the pioneering studies on the lac operon, it has been well established that intermediary metabolites serve as signaling molecules in bacteria and yeast (Gancedo, Microbiol. Mo. Biol. Rev., 62:334-361 (1998); Ullmann, Biochimie, 67:29-34 (1985)). Understanding of metabolite control in mammalian cells has been hampered by the need to identify metabolic signals and their cognate sensors.

Previous studies on the bile acid-signaling pathway in liver were performed using intact animals or cultured hepatocytes. Workers using studies of this design were not able to discover which compounds were the ultimate bile acid signaling molecules because the bile acids were subject to metabolic conversion in these systems by intestinal microorganisms and/or liver-specific enzymes into a number of different products. The receptor which acts as a sensor for cholesterol and bile acid signals thus had not been identified.

Because inhibition of cholesterol catabolism by bile acids limits the amount of cholesterol that can be excreted as bile acids, identification of the nuclear receptor which regulates bile acid and cholesterol metabolism would be a major advantage in developing treatment modalities for individuals with hypercholesterolemia or other conditions related to activation levels of a gene regulated by this receptor. The current inability to interfere with the transcription or expression of bile acid regulated genes, for example the negative feedback imposed by bile acids on cholesterol catabolism, poses a serious stumbling block in treating individuals with conditions related to these genes, for example hypercholesterolemia. Any condition involving a defect in the regulation of a bile acid nuclear receptor controlled gene would be ameliorated by modification of the receptor activity. The nuclear bile acid sensor has been shown to respond to bile acids by either stimulating or suppressing target gene transcription. These activities are mediated by positive FXR response elements within these genes. For example, bile acids coordinately repress the transcription of the liver- and ileal/renal-specific bile acid transporters. (Torchia et al., Biochem. Biophys. Res. Commun., 225:128-133 (1996), sterol 27-hydroxylase (Cyp27) (Twisk et al., Biochem. J., 305:505-511 (1995) and sterol 12α-hydroxylase (Cypl2) (Einarsson et al., J. Lipid Res., 33:1591-1595 (1992)). Therefore, detrimental metabolic conditions which could be ameliorated by either stimulation or suppression of a bile acid receptor target gene may be treated with bile acid receptor ligands.

There is consequently a need for compounds and methods to modulate the expression of genes regulated by bile acids such as Cyp7a. A further need exists for a screening method for identifying compounds that can provide a pharmacologic intervention to modify the regulation of transcription of bile acid regulated genes. Such compounds and methods would be of value to patients who would benefit from modification of bile acid regulated gene transcription, such as individuals suffering from hypercholesterolemia.

### SUMMARY OF THE INVENTION

Accordingly, the invention provides a method of modulating an FXR-dependent physiological process which comprises modulating the activation of FXR. The physiological process may be cholesterol metabolism. Preferred methods include those which comprise modulating expression of an FXR target gene, for example, the gene which encodes Cyp7a, Cyp8b, phospholipid transfer protein (PLTP), ileal bile acid binding protein (IBABP), sodium taurocholate cotransporter protein (Ntcp), liver fatty acid binding protein (L-FABP) and bile salt export pump (Bsep).

Methods of modulating an FXR dependent physiological process include those wherein cholesterol metabolism is increased by upregulating expression of the gene encoding Cyp7a to a level of expression that is substantially more than which occurs naturally in the cell. This upregulation of expression of the gene encoding Cyp7a may be achieved by inhibiting activation of FXR such as with an antagonist or a blocking compound. Methods wherein cholesterol metabolism is decreased are also provided by the invention by downregulating expression of the gene encoding Cyp7a to a level that is substantially less than that which occurs naturally in the cell. Downregulation of expression of the gene encoding Cyp7a maybe achieved by increasing activation of FXR such as by application of an FXR agonist.

Upregulation or downregulation of the genes encoding Cyp8b phospholipid transfer protein, ileal bile acid binding protein, sodium taurocholate cotransporter protein, liver fatty acid binding protein or bile salt export pump may be achieved in the same way, affecting their respective metabolic processes, such as bile acid uptake, bile acid secretion from the liver and regulation of triglyceride levels.

The invention also provides methods of modulating the FXR dependent physiologic process of triglyceride metabolism. In these methods, triglyceride levels are decreased by increasing activation of FXR and increased by inhibiting activation of FXR.

The invention also provides methods which comprise contacting FXR with a compound according to Formula I:

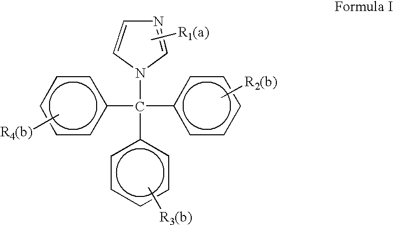

wherein (a) represents an integer from 0 to 3 and each (b) may be the same or different and represents an integer from 0 to 5;

wherein each R₁, R₂, R₃, and R₄ may be the same as or different from any other R₁, R₂, R₃ or R₄ and represents a moiety selected from the group consisting of C₁₋₄ alkyl, C₁₋₄ alkenyl, aryl, alkylaryl, halo, trihalomethyl, furanyl, thiophenyl, pyrrolyl, pyrazolyl, diazolyl, triazolyl, tetrazolyl, dithiolyl, oxathiolyl, oxazolyl, isoxazolyl, thiazolyl, isothiazolyl, oxadiazolyl, oxatriazolyl, dioxazolyl, isoxazinyl and piperazinyl, and

wherein said moiety may be unsubstituted or substituted with one or more substituent selected from the group consisting from methyl, ethyl, amino, halo, trihalomethyl and nitro. Compounds of Formula I which may be useful include clotrimazole, Compound A and Compound B, the structures of which are shown in Table II, below.

The invention also provides methods for screening for pharmacologically active compounds which comprise determining whether a compound activates or inhibits activation of the FXR receptor. Such methods can be used for screening for compounds capable of modulating an FXR-dependent physiological process selected from the group consisting of cholesterol metabolism and triglyceride metabolism. Methods may include those which involve determining whether the compound inhibits FXR activation, thereby increasing cholesterol catabolism.

The invention also provides methods of screening compounds useful in modulating FXR-mediated gene transcription which comprise contacting a mixture of FXR and RXR with a compound and determining whether the compound promotes interaction between FXR-RXR heterodimer and coactivator or between FXR and coactivator.

In a further embodiment, the invention also provides methods of screening compounds for FXR antagonist activity which comprise contacting a mixture of FXR and RXR and a known FXR agonist with at least one of the compounds and determining whether the compound inhibits the agonist-promoted activation of an FXR-RXR heterodimer. Known FXR agonists which may be useful in the methods include compounds of Formula I:

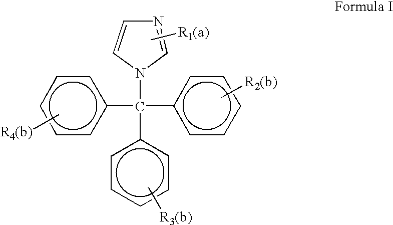

wherein (a) represents an integer from 0 to 3 and each (b) may be the same or different and represents an integer from 0 to 5;

wherein each R₁, R₂, R₃, and R₄ may be the same as or different from any other R₁, R₂, R₃ or R₄ and represents a moiety selected from the group consisting of C₁₋₄ alkyl, C₁₋₄ alkenyl, aryl, alkylaryl, halo, trihalomethyl, furanyl, thiophenyl, pyrrolyl, pyrazolyl, diazolyl, triazolyl, tetrazolyl, dithiolyl, oxathiolyl, oxazolyl, isoxazolyl, thiazolyl, isothiazolyl, oxadiazolyl, oxatriazolyl, dioxazolyl, isoxazinyl and piperazinyl, and

wherein said moiety may be unsubstituted or substituted with one or more substituent selected from the group consisting from methyl, ethyl, amino, halo, trihalomethyl and nitro. Compounds such as, for example, clotrimazole, Compound A and Compound B may be useful for these methods.

The methods include those in which the RXR is an RXR mutant (RXRm) which contains a functional DNA-binding domain and which has a mutation in the ligand-binding domain which prevent substantial activation by RXR ligands but which does not otherwise substantially affect the ability of the RXR mutant receptor to form heterodimers with FXR.

In a further embodiment the invention provides methods for screening compounds for cholesterol catabolism-modulating activity which comprise (1) providing a first mixture which contains an FXR receptor, an RXR receptor, and a labeled DNA probe which contains a sequence of nucleotides to which the DNA-binding domain of a ligand-FXR-RXR complex specifically binds; (2) providing a second mixture which contains an FXR receptor, an RXR mutant receptor (RXRm) which contains a functional DNA-binding domain and which has a mutation in the ligand-binding domain which prevents substantial activation by RXR ligands but which does not otherwise substantially affect the ability of the RXR mutant receptor to form heterodimers with FXR or such heterodimers to recruit coactivator, and a labeled DNA probe which contains a sequence of nucleotides to which the DNA-binding domain of a ligand-FXR-RXR complex specifically binds; (3) contacting the first and second mixtures with the compound being screened; (4) determining whether the compound causes interaction of an FXR-RXR heterodimer with coactivator or of FXR with coactivator; and (5) determining whether the compound being screened causes interaction of an FXR-RXRm heterodimer with coactivator.

Such methods may further comprise contacting the first and second mixtures with a known FXR ligand and selecting compounds that inhibit the ability of the known FXR ligand to cause the FXR-RXR-heterodimer or FXR to interact with coactivator. Known FXR agonists which may be useful in the methods include compounds of Formula I:

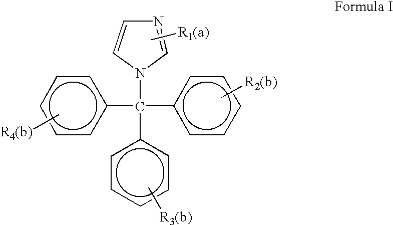

wherein (a) represents an integer from 0 to 3 and each (b) may be the same or different and represents an integer from 0 to 5;

wherein each R₁, R₂, R₃, and R₄ may be the same as or different from any other R₁, R₂, R₃ or R₄ and represents a moiety selected from the group consisting of C₁₋₄ alkyl, C₁₋₄ alkenyl, aryl, alkylaryl, halo, trihalomethyl, furanyl, thiophenyl, pyrrolyl, pyrazolyl, diazolyl, triazolyl, tetrazolyl, dithiolyl, oxathiolyl, oxazolyl, isoxazolyl, thiazolyl, isothiazolyl, oxadiazolyl, oxatriazolyl, dioxazolyl, isoxazinyl and piperazinyl, and

wherein said moiety may be unsubstituted or substituted with one or more substituent selected from the group consisting from methyl, ethyl, amino, halo, trihalomethyl and nitro. Compounds such as, for example, clotrimazole, Compound A and Compound B may be useful in the methods. In addition, the methods may further comprise selecting compounds that do not cause substantial interaction of FXR, the FXR-RXR heterodimer or the FXR-RXRM heterodimer with coactivator.

In a further embodiment, the invention provides methods of screening for compounds useful in modulating FXR-mediated gene transcription which comprise contacting a mixture of FXR, RXR and an FXR/RXR coactivator with a compound and determining whether the compound promotes coactivator recruitment by an FXR-RXR heterodimer.

In yet a further embodiment the invention provide methods of screening compounds for FXR antagonist activity which comprise contacting a mixture of FXR, RXR, an FXR/RXR coactivator and a known FXR agonist with at least one of the compounds and determining whether the compound inhibits the agonist-promoted coactivator recruitment by an FXR-RXR heterodimer. Known FXR agonists which may be useful in these methods include compounds of Formula I:

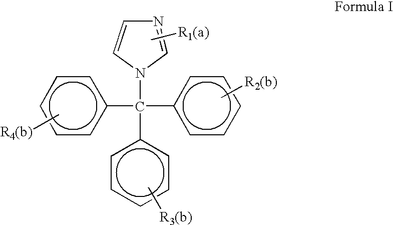

wherein (a) represents an integer from 0 to 3 and each (b) may be the same or different and represents an integer from 0 to 5;

wherein each R₁, R₂, R₃, and R₄, may be the same as or different from other R₁, R₂, R₃, or R₄ and represents a moiety selected from the group consisting of C₁₋₄ alkyl, C₁₋₄ alkenyl, aryl, alkylaryl, halo, trihalomethyl, furanyl, thiophenyl, pyrrolyl, pyrazolyl, diazolyl, triazolyl, tetrazolyl, dithiolyl, oxathiolyl, oxazolyl, isoxazolyl, thiazolyl, isothiazolyl, oxadiazolyl, oxatriazolyl, dioxazolyl, isoxazinyl and piperazinyl, and wherein said moiety may be unsubstituted or substituted with one or more substituent selected from the group consisting from methyl, ethyl, amino, halo, trihalomethyl and nitro. Clotrimazole, Compound A and Compound B are examples of compounds which may be used in this method.

Preferred methods include those wherein the RXR is an RXR mutant (RXRm) which contains a functional DNA-binding domain and which has a mutation in the ligand-binding domain which prevents substantial activation by RXR ligands but which does not otherwise substantially affect the ability of the RXR mutant receptor to form heterodimers with FXR or of such heterodimers to recruit coactivator. Preferred methods also include those in which the coactivator is a polypeptide or active fragment thereof which contains a peptide motif that interacts with FXR, the FXR-RXR heterodimer or the FXR-RXRm heterodimer in a ligand-dependent manner. Suitable coactivators for use in the method include SRC-1, GRIP-, (GRIP1), ACTR and PBP/DRIP205/TRAP220. Suitable DNA probes for use in the method include those of SEQ ID NO: 3 and SEQ ID NO: 4.

In a further embodiment, the invention provides methods of screening for compounds useful in modulating FXR-mediated gene transcription, which comprise (a) transfecting mammalian cells with a gene encoding FXR under control of an operative promoter; (b) transfecting the cells with an operative reporter gene under control of a promoter linked to a DNA sequence which encodes an operative response element to which ligand-activated FXR or FXR complex binds to initiate transcription of the reporter gene; (c) culturing the cells in the presence of a compound being screened; and (d) monitoring the cells for transcription or expression of the reporter gene as an indication of FXR activation. Methods also include those in which the cells are cultured in the presence of a known FXR ligand and the diminution of transcription or expression of the reporter gene is an indication that the compound being screened is an FXR antagonist. Known FXR ligands which may be useful in these methods include compounds of Formula I:

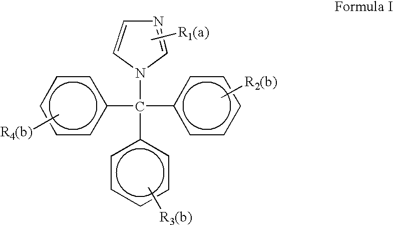

wherein (a) represents an integer from 0 to 3 and each (b) may be the same or different and represents an integer from 0 to 5;

wherein each R₁, R₂, R₃, and R₄, may be the same as or different from other R₁, R₂, R₃, or R₄ and represents a moiety selected from the group consisting of C₁₋₄ alkyl, C₁₋₄ alkenyl, aryl, alkylaryl, halo, trihalomethyl, furanyl, thiophenyl, pyrrolyl, pyrazolyl, diazolyl, triazolyl, tetrazolyl, dithiolyl, oxathiolyl, oxazolyl, isoxazolyl, thiazolyl, isothiazolyl, oxadiazolyl, oxatriazolyl, dioxazolyl, isoxazinyl and piperazinyl, and

wherein said moiety may be unsubstituted or substituted with one or more substituent selected from the group consisting from methyl, ethyl, amino, halo, trihalomethyl and nitro. Clotrimazole, Compound A and Compound B are compounds which may be of use. These methods may be used to identify compounds that are useful for increasing cholesterol catabolism.

In a further embodiment, the invention provides non-naturally occurring or natural compounds selected by any of the methods described above. In yet further embodiments the invention provides pharmaceutical compositions comprising a therapeutically or prophylactically effective amount of a compound of compounds selected according to the methods described above in combination with a pharmaceutically acceptable carrier.

In yet a further embodiment the invention provides a method for treating a mammal for hypercholesterolemia which comprises administering an effective amount of any of the pharmaceutical compositions described above.

### BRIEF DESCRIPTION OF THE DRAWINGS

FIG. 1 provides data from an activation assay of FXR-RXR heterodimers and several other orphan receptors with bile extract. The receptors, each of which is known in the art and is further identified herein by its Genbank accession number are CARβ, PPARα, PPARδ, TR2-11, LXRα, GCNF, SF1, RORα, Nurr1, DAX, and ERR2.

FIG. 2 provides data comparing the activation by a synthetic specific RXR ligand, LG268, of chimeric receptors containing the yeast GAL4 DNA binding domain fused to the wild-type RXR ligand binding domain or a mutant RXR (RXRm) ligand binding domain.

FIG. 3 is an autoradiogram showing results of electrophoretic mobility experiments demonstrating the formation of receptor-coactivator complexes with wild-type RXR (RXR-CoA) or mutant RXR (RXRm-CoA) at increasing concentrations of the RXR ligand, LG268.

FIG. 4 provides quantitative data for the results shown in FIG. 3.

FIG. 5 provides data comparing activation of RXR and FXR heterodimers by the RXR ligand LG268 and a bile extract.

FIG. 6 shows FXR, RXR and RXRm activation data of fractions obtained from preparative thin layer chromatography of bile extract.

FIG. 7 shows the reversed phase HPLC absorbance tracing of fraction B from FIG. 6.

FIG. 8 provides data showing that HPLC peak Z from FIG. 7 potently activated FXR-RXRm but has no effect on RXR.

FIG. 9 shows the level of FXR activation by several different free bile acids. Juvenile hormone III and the RXR ligand LG268 were included as controls. UDCA indicates ursodeoxycholic acid (5β-cholanic acid-3α, 7β-diol).

FIG. 10 shows the level of FXR activation by CA, CDCA, DCA and LCA, unconjugated or conjugated with either glycine or taurine, in the presence or absence of a liver bile acid transporter.

FIG. 11 provides FXR activation dose-response data for CDCA, DCA and LCA. The EC₅₀ for each of the compounds was approximately 50 μM.

FIG. 12 summarizes structure-activity information derived from the data given in the previous Figures. ++ indicates >200-fold activation and + indicates 100-150-fold activation of FXR-RXR heterodimers.

FIG. 13 provides data showing that CDCA and LCA both activate transcription in cells co-expressing a GAL-L-FXR chimera and the RXR LBD (L-RXR).

FIG. 14 shows data demonstrating activation after recruitment of a GAL-4 coactivator fusion protein (GAL-CoA) which was dependent on the presence of both the RXR and FXR LBDs in a mammalian two-hybrid assay.

FIG. 15 shows electrophoretic mobility data demonstrating coactivator recruitment by different ligands in the presence of FXR and RXR or FXR and RXRm.

FIG. 16 provides data showing inhibition of FXR by three azole compounds according to Formula I.

### DETAILED DESCRIPTION OF THE INVENTION

This invention provides a method for modulating the transcription of genes regulated by the bile acid nuclear receptor (BAR) which has been identified as the FXR receptor. The invention also provides a method for identifying compounds which activate or inhibit FXR and are useful in the method.

It has been found that extracts of bile specifically activate the orphan receptor FXR. An active endogenous bile acid signaling molecule which activates this receptor was purified to homogeneity and identified as chenodeoxycholic acid (CDCA). Further analysis and structure-activity studies revealed that CA, DCA and LCA also activate FXR. Additionally, LCA was found to promote coactivator recruitment in vitro.

To verify that a bile acid component was able to bind to and activate the FXR orphan receptor, an extract of porcine bile (Sigma) was prepared and tested on a number of orphan nuclear receptors. The receptors to be tested were expressed in CV-1 cells, which are derived from COS cells. The cells are described in Boyer et al., Am. J. Physiol, 266:G382-G387 (1994).

Use of a standard model heterologous cell system to reconstitute bile acid responsiveness allows activity to be monitored in the absence of the metabolic events which may obscure the process being tested. Any suitable heterologous cell system may be used to test the activation of potential or known bile acid nuclear receptor ligands, as long as the cells are capable of being transiently transfected with the appropriate DNA which expresses receptors, reporter genes, response elements, and the like. Cells which constitutively express one or more of the necessary genes may be used as well. Cell systems that are suitable for the transient expression of mammalian genes and which are amenable to maintenance in culture are well known to those skilled in the art.

TABLE I
Reporter/Receptor Pairs for
Orphan Receptor Activation
Assay

| No. | Reporter | Receptor(s) |
|---|---|---|
| 1 | EcRE × 6 | FXR + RXR |
| 2 | βRE2 × 3 | CARβ |
| 3 | PPRE × 3 | PPARα, δ, |
| | | TR2-11 |
| 4 | LXRE × 3 | LXRα |
| 5 | DR0 × 2 | GNCF |
| 6 | SF1 × 4 | SF1 |
| 7 | UAS_{G} × 4 | GAL-RORα, |
| | | GAL-Nurr1, |
| | | GAL-DAX, |
| | | GAL-ERR2 |

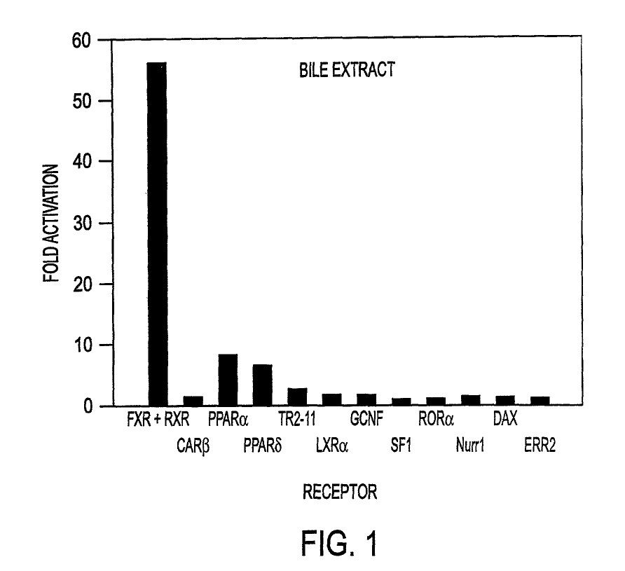

To test the activation of various orphan receptors by bile acids, CV-1 cells were transiently transfected with expression vectors for the receptors indicated in FIG. 1 along with appropriate reporter constructs according to methods known in the art. Suitable reporter gene constructs are well known to skilled workers in the fields of biochemistry and molecular biology. Reporter/receptor pairs used in the assay reported in FIG. 1 are listed in Table I. All transfections additionally contained CMV-βgal as an internal control. Suitable constructs for use in the these studies may conveniently be cloned into pCMV. pCMV contains the cytomegalovirus promoter/enhancer followed by a bacteriophage T7 promoter for transcription in vitro. Other vectors known in the art can be used in the methods of the present invention.

Genes encoding the following full-length previously described proteins, which are suitable for use in the studies described herein, were cloned into pCMV: rat FXR (accession U18374), human RXRα (accession X52773), human TRβ (accession X04707, human LXRα (accession U22662), mouse PPARα (accession X57638), mouse PPARΔ (accession U10375), human TR2-11 (accession M29960), mouse GCNF (accession u14666), mouse SF1 (accession S65878). All accession numbers in this application refer to GenBank accession numbers. GAL4 fusions containing receptor fragments were constructed by fusing the following protein sequences to the C-terminal end of the yeast GAL4 DNA binding domain (amino acids 1-147) from pSG424 (Sadowski and Ptashne, Nucl. Acids Res., 17:7539 (1989)): GAL-L-RXR (human RXRα Glu 203—Thr 462), GAL-L-FXR (rat FXR LBD Leu 181—Gln 469), GAL-RORα (human RORα1 Arg 140—Gly 523, accession U04897), GAL-Nurr1 (mouse Nurrl, Cys 318—Phe 598, accession S53744), GAL-DAX (human DAX-1, accession U31929), GAL-ERR2 (human ERR2, Glu 171—Val 433, accession X51417), GAL-CoA (human SRC-1 Asp 617—Asp 769, accession U59302).

The RXR LBD expression construct L-RXR contains the SV40 TAg nuclear localization signal (APKKKRKVG (SEQ ID NO: 1)) fused upstream of the human RXRα LBD (Glu 203—Thr 462). VP-L-FXR contains the 78 amino acid Herpes virus VP16 transactivation domain linked to the amino terminal end of the rat FXR LBD (Leu 181—Gln 469). CMV-βgal, used as a control gene for comparison with the activation of the receptor or receptor domain being tested, contains the E. coli β-galactosidase coding sequences derived from pCH110 (accession U02445). This gene was conveniently used here, however any unrelated gene which is available and for which a convenient assay exists to measure its activation may be used as a control with the methods of this invention.

To determine which orphan receptor or receptors were activated by bile and could be exogenously manipulated to modify transcription of bile acid responsive genes, the transfected cells were treated with porcine bile extract. The bile extract was prepared as follows. Bile (Sigma, 1 g) was dissolved in water and adjusted to pH 4.0. The water-insoluble material was further extracted with methanol. Methanol-soluble material was dried and redissolved at 100 μg/ml.

CV-1 cells for the activation assays were grown in Dulbecco's modified Eagle's medium supplemented with 10% resin charcoal-stripped fetal bovine serum, 50 U/ml penicillin G and 50 μg/ml streptomycin sulfate (DMEM-FBS) at 37° C. in 5% CO₂. One day prior to transfection, cells were plated to 50-80% confluence using phenol red free DMEM-FBS.

The cells were transiently transfected by lipofection. Reporter constructs (300 ng/10⁵ cells) and cytomegalovirus-driven expression vectors (20-50 ng/10⁵ cells) were added, with CMV-β-gal (500 ng/10⁵ cells) as an internal control. After 2 hours, the liposomes were removed and the cells were treated for approximately 45 hours with phenol red free DMEM-FBS containing the test bile acid and other compounds.

Any compound which is a candidate for activation of FXR may be tested by this method. Generally, compounds are tested at several different concentrations to optimize the chances that activation of the receptor will be detected and recognized if present. After exposure to ligand, the cells were harvested and assayed for β-galactosidase activity (control) and activity of the specific reporter gene. All assays disclosed here were performed in triplicate and varied within experiment less than 15%. Each experiment was repeated three or more times with similar results.

Activity of the reporter gene can be conveniently normalized to the internal control and the data plotted as fold activation relative to untreated cells. See FIG. 1 for data showing the activation of orphan receptors by bile extract. As shown in the Figure, the bile extract was a strong activator (56-fold) of FXR but had little or no effect on the other orphan receptors tested.

As discussed above, FXR binds to its response element as a heterodimer with RXR (9-cis retinoic acid receptor). This heterodimer can be activated by FXR-binding ligands or by RXR-binding ligands (Forman et al., Cell 803-812 (1995); Zavaki et al., Proc. Natl. Acad. Sci. (USA), 94:7909-7914 (1997)). Because the activation of the FXR-RXR heterodimers by bile extract could reflect the presence of ligands for either FXR or RXR, an FXR-RXR complex that is defective in its response to RXR ligands was created to screen for FXR-specific activators. RXRm is a human 9-cis retinoic acid receptor ligand binding domain which contains a single point mutation (Asp 322→Pro) . This mutated receptor domain retains the ability to bind to DNA and to form heterodimeric complexes with FXR, however it lacks the ability to respond to low concentrations of ligand as the wild-type receptor domain does. The availability of this defective RXR ligand-binding domain permits the creation of a screening assay which detects activation of the bile acid nuclear receptor in the absence of RXR effects, preventing false positive results which would otherwise occur.

For an RXR mutant to function in this procedure, the receptor should be minimally activated by RXR ligands and fail to recruit coactivator when exposed to RXR ligands, but retain the ability to dimerize with FXR and to bind DNA as a heterodimer with FXR. Finally, the mutant should not substantially interfere with the normal activity of the bile acid nuclear receptor. To ensure these qualities, tests were performed on the mutant ligand binding domain as described below. Other mutants also can be tested in the same way to determine their suitability for use in the methods of this invention.

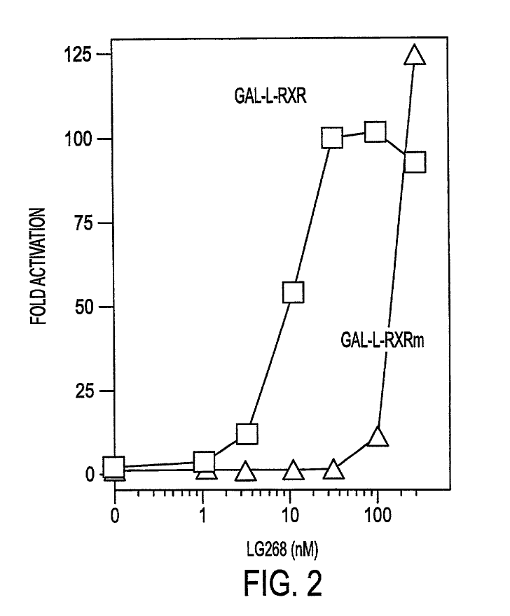

An RXR mutant (RXRm) containing a single point mutation in the LBD (Asp 322→Pro) has been found to function particularly well in these analyses. Chimeric receptors containing the yeast GAL4 DNA binding domain fused to the ligand-binding domain of either wild-type RXR (GAL-L-RXR) or RXRm (GAL-L-RXRm) were tested for a response to a synthetic RXR-specific ligand (LG268, 6-[l-(3,5,5,8,8-pentamethyl-5,6,7,8-tetrahydronaphthalen-2-yl)cyclopropyl]nicotinic acid) in the same way as described for the data in FIG. 1 for activation of the orphan receptors by bile acids. CV-1 cells were transiently transfected with a UAS_{G}×4 reporter and expression vectors for β-galactosidase and either GAL-L-RXR or the GAL-L-RXRm LBD mutant. After transfection, cells were treated with the concentrations of LG268 indicated in FIG. 2. Dimers having the mutant RXR ligand binding domain demonstrated a 10-fold decrease in their potency of activation over dimers having a wild-type RXR ligand binding domain. See FIG. 2. Similar results were observed with full-length RXR and RXRm receptors (data not shown).

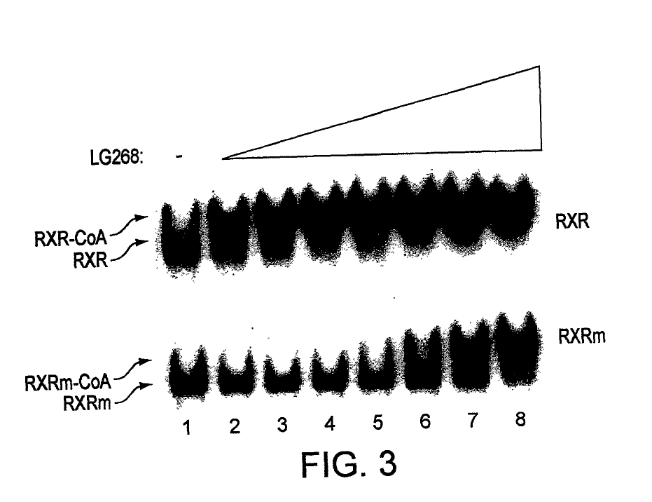

To further confirm the suitability of this mutant, the ability of wild-type RXR and RXRm to bind DNA and recruit coactivator in response to ligand was compared. Electrophoretic mobility shift experiments were performed by first incubating together a mixture of 1.2 μl in vitro translated RXR (FIG. 3, top panel) or RXRm (FIG. 3, bottom panel), 5 μg of purified recombinant GST-GRIP1 coactivator (described below), and a ³²P-labeled DR1 probe (5′-AGCTACCAGGTCAAAGGTCACGTAGCT-3′; SEQ ID NO: 2) with increasing amounts of the RXR ligand LG268 (0-1000 nM). The DR1 probe of SEQ ID NO: 2 was used for all RXR homodimer tests disclosed here. Any nucleic acid probe which is substantially homologous to the DNA-binding domain target sequence may be used for such assays, as long as the ligand-occupied heterodimer binds to the probe with sufficient avidity for the detection method used. Likewise, any convenient label for the nucleotide probe sensitive enough to detect the presence of complexes in the mixture is contemplated for use with the inventive methods.

During incubation, complexes form in which dimers recruit coactivator and bind to the labeled DNA probe. After incubation, the mixture is subjected to electrophoresis under nondenaturing conditions. For this assay, the complexes were electrophoresed through a 5% polyacrylamide gel in 45 mM Tris-base buffer, containing 45 mM boric acid and 1 mM EDTA at room temperature. The gel was subjected to autoradiography to detect the labeled complexes and other components.

In FIG. 3, CoA indicates a GST-fusion containing the three receptor interaction domains from the coactivator GRIP1. The electrophoretic mobility shift results indicate that RXRm recruits coactivator with a 100-fold decrease in potency compared to wild type RXR. While both mutant and wild-type receptors bound DNA (FIG. 3, lane 1), RXRm failed to recruit coactivator at ligand concentrations that were sufficient for maximal recruitment by the wild-type receptor (FIG. 3, compare upper and lower panels, lanes 2-6).

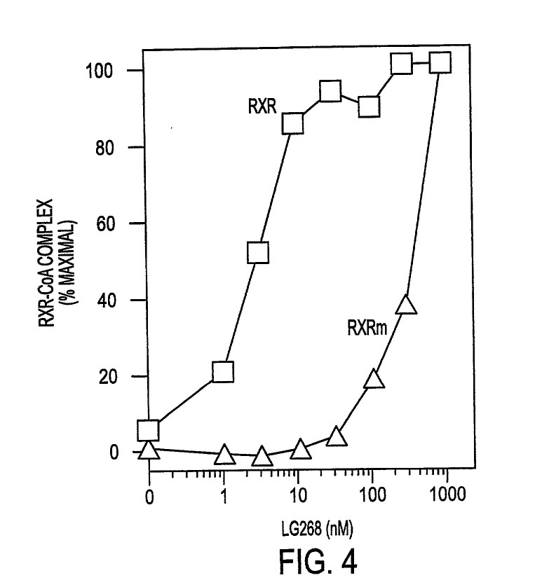

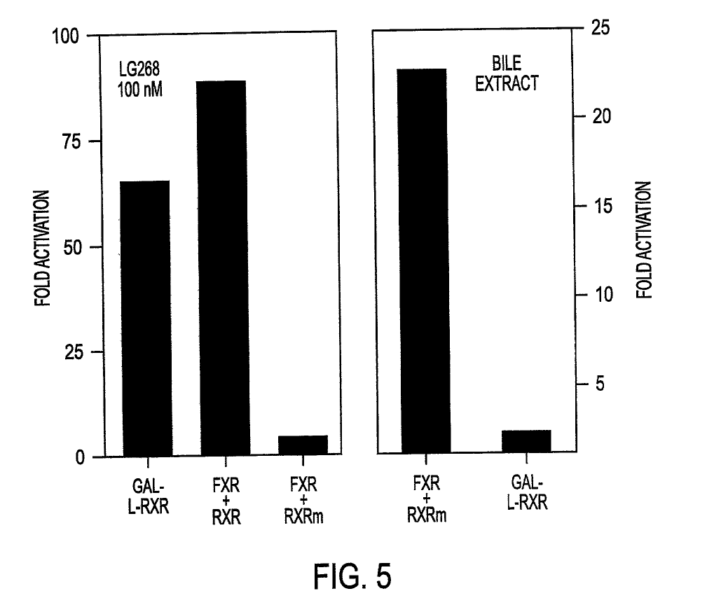

Quantitation of the amount of RXR-coactivator complex shown in FIG. 3 was determined by phosphorimager analysis of the autoradiogram and plotted as a function of the LG268 concentration. The data indicated that recruitment of coactivator by RXRm required approximately 100-fold higher concentrations of ligand (FIG. 4). Since the RXR mutant retained the ability to bind DNA as a heterodimer with FXR (FIG. 5 and data not shown), but lacked the ability to respond strongly to low concentrations of ligand, FXR-RXRm heterodimers could be used to verify activation of the FXR subunits in tests with various ligands. Such heterodimers therefore may advantageously be employed in a method to screen for compounds which activate FXR and thus for compounds able to modify transcription of genes regulated by the receptor.

Confirmation of the analytical procedure was achieved by testing FXR-RXR and FXR-RXRm dimers for activation by RXR and FXR ligands. CV-1 cells were transfected with plasmids harboring the receptor domains indicated in FIG. 5 and treated with either 100 nM LG268 (left panel) or a methanol extract of porcine bile (200 μg/ml, right panel). While LG268 activated RXR (GAL-L-RXR) and FXR-RXR heterodimers, FXR-RXRm showed little or no response to the RXR-specific ligand LG268 (FIG. 5, left panel). In contrast, the bile extract retained the ability to activate FXR-RXRM but had little effect on GAL-L-RXRm (FIG. 5, right panel). Similar results were obtained when RXRm was replaced with a different RXR mutant containing a defective AF2 transactivation domain (Phe 450→Ala, Schulman et al., Mol. Cell. Biol., 16:3807-3813 (1996)) (data not shown). This type of assay therefore can specifically discriminate between activation by RXR ligands and FXR ligands. These particular data indicate that bile extract contains an FXR-specific activator.

Further data showed that activation requires the AF2 transactivation domain of FXR (data not shown). In addition, bile acids induced activation with the expected kinetics (activity is observed within one hour of ligand addition to cells; data not shown). Taken together, the data provided herein demonstrate that FXR is the endogenous bile acid sensor which can be manipulated exogenously with appropriate ligands to modify the regulation of genes dependent on activation via FXR, such as important genes involved in the control of cholesterol metabolism.

A chemical fractionation scheme was devised to identify and purify the biliary component in the bile extract which binds to and activates FXR. As an initial step, the methanol-water bile extract was fractionated by silica gel chromatography. Briefly, the extract was applied to a column and successively eluted with chloroform-methanol at ratios of 8:1 and 4:1, then with 100% methanol. Fifty-six fractions were collected, pooled and tested for their ability to activate FXR-RXRm. The active fraction was further purified by preparative thin layer chromatography (PTLC) and separated into 5 fractions (A-E).

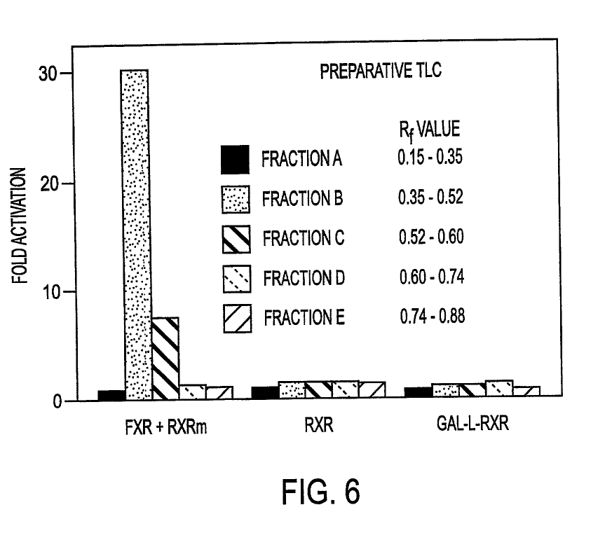

To test for FXR activation by material in these fractions, CV-1 cells were transfected with plasmids harboring the receptor domains indicated in FIG. 6 and treated with 25 μg/ml of each of the 5 PTLC fractions. Fraction B (PTLC 0.35<R_{f}<0.52) was the most active (30-fold greater activation of FXR-RXRm relative to activation of RXR). See FIG. 6.

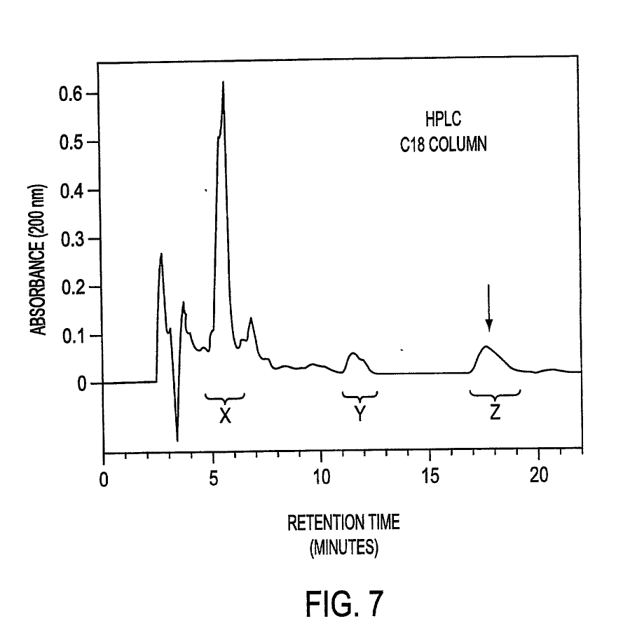

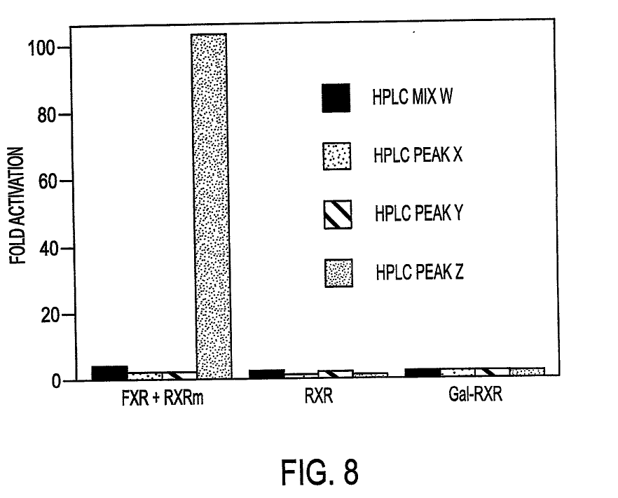

The active material of PTLC fraction B was further purified by reverse-phase HPLC on a C18 column. Absorbance was monitored at 200 nm. Three main peaks were resolved (peaks X, Y and Z; FIG. 7). These three peaks were collected in isolation. The remaining fractions were pooled to form a fourth fraction (W). The four fractions were tested for activation of FXR-RXRm as above. Briefly, CV-1 cells were transfected with plasmids harboring the receptor domains indicated in FIG. 8 and treated with each of the HPLC fractions at concentrations of 25 μg/ml. Fractions W, X and Y had little or no activity (FIG. 8). Remarkably, peak Z induced a dramatic 102-fold activation of FXR-RXRm but had no effect on RXR. These data indicate that the material in peak Z not only potently activated FXR, but did not contain any RXR activating material. Thus, the material in peak Z was selected for structural analysis.

After methylation of the material in peak Z, tandem gas chromatography-mass spectrometry (GC-MS) was performed. The gas chromatogram indicated that peak Z contained one predominant peak, indicating that the active component had been purified to near homogeneity. Compound Z had a retention time of 14.41 minutes in this assay and was indistinguishable from a synthetic chenodeoxycholic acid (CDCA) standard. The mass-spectrum of the material of peak Z also was indistinguishable from that of a chenodeoxycholic acid (CDCA) standard. To further confirm the identity of this material, ¹³C-NMR, ¹H-NMR, DEPT, DQFCOSY and HMQC spectra (data not shown) were obtained and found to be identical to the CDCA standard. The component in porcine bile extract which activates the bile acid nuclear receptor in this assay therefore was identified as CDCA.

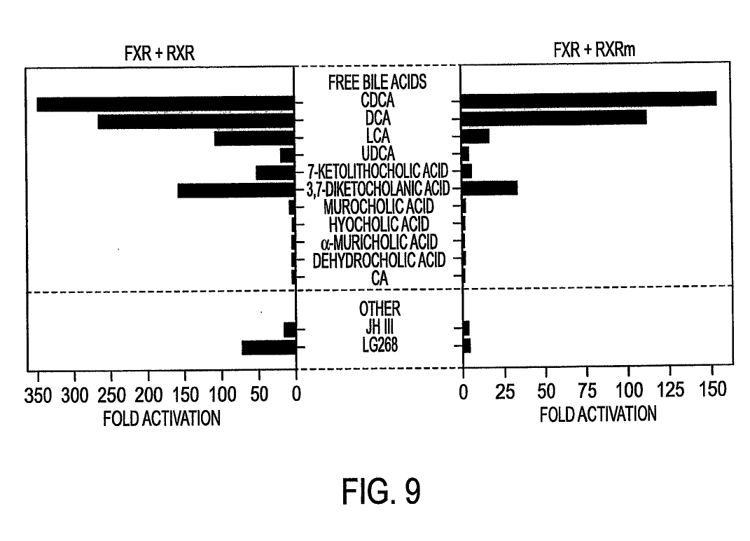

A variety of commercially available bile acids (Sigma) were tested for their ability to activate known FXR-RXR (FIG. 9; left panel) and FXR-RXRm (FIG. 9, right panel). The RXR ligands LG268 and juvenile hormone III were also included as test ligands for comparison. For these assays, CV-1 cells were transfected with an EcRE×6 reporter and expression vectors for β-galactosidase and FXR+RXR (left panel) or FXR+RXRm (right panel) and treated with the indicated bile acids (100 μM), juvenile hormone III (JH III, 50 μM) or LG268 (100 nM). Bile acids are denoted in the Figure as follows: CA, cholic acid; CDCA, chenodeoxycholic acid; DCA, deoxycholic acid; LCA, lithocholic acid; UDCA, ursodeoxycholic acid. As expected from the studies of bile acid extract, synthetic CDCA proved to be a highly effective activator of FXR (346-fold activation; FIG. 9, left panel). CDCA failed to activate other receptors, including RXRα; PPARα, γ and δ; VDR; T₃Rβ; RAR; PXR; LXRα and CARβ (data not shown).

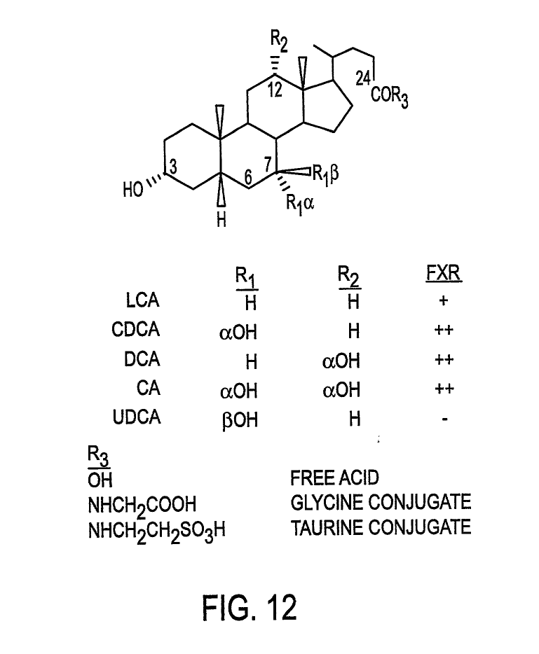

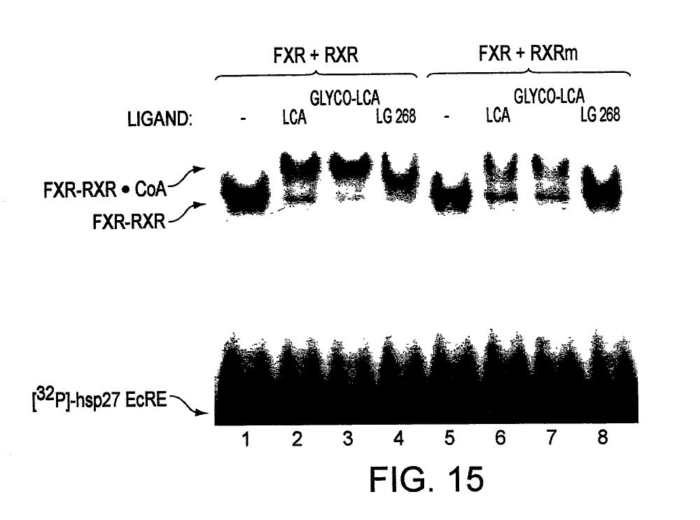

The secondary bile acids, CDA and LCA, were also highly effective, inducing 246- and 106-fold activations of FXR-RXR, respectively. Qualitatively similar results were seen with FXR-RXRm (FIG. 9, right panel), indicating that all of these bile acids act through the FXR subunit. Ursodeoxycholic acid (UDCA, 5β-cholanic acid-3α,7β-diol), the 7β-epimer of CDCA, was inactive while substitution of a hydroxyl group with a ketone at the 7-position produced a compound (7-ketolithocholic acid, 5β-cholanic acid-3α-ol-7-one) with activity intermediate between CDCA and UDCA FIGS. 9, 15). Thus, the configuration around the 7 position is a crucial determinant of FXR activity with 7α-OH >7-keto>>7β-OH. Comparison of 7-ketolithocholic acid and 3,7-diketocholanic acid (5β-cholanic acid-3,7-dione) suggests that a ketone in the 3-position is preferred to a 3α-hydroxyl group. Several di- and tri-hydroxy bile acids with a hydroxyl group in the 6 position were inactive (murocholic acid, hyocholic and α-muricholic acid) as was dehydrocholic acid (5β-cholanic acid-3,7,12-trione). Taken together, these data suggest that 3,7- and 3,12-substituted bile acids are highly effective activators of FXR. FIG. 12 is a summary comparison of the chemical structure of key bile acids and their efficacy as FXR activators.

TABLE II
Chemical Structures of Azole Antagonists of FXR.

| | |
|---|---|
| 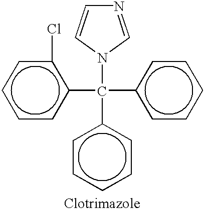 | 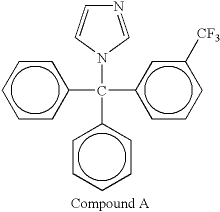 |
| 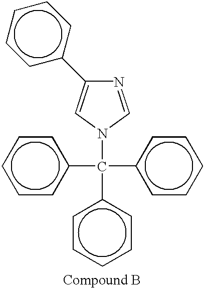 | |

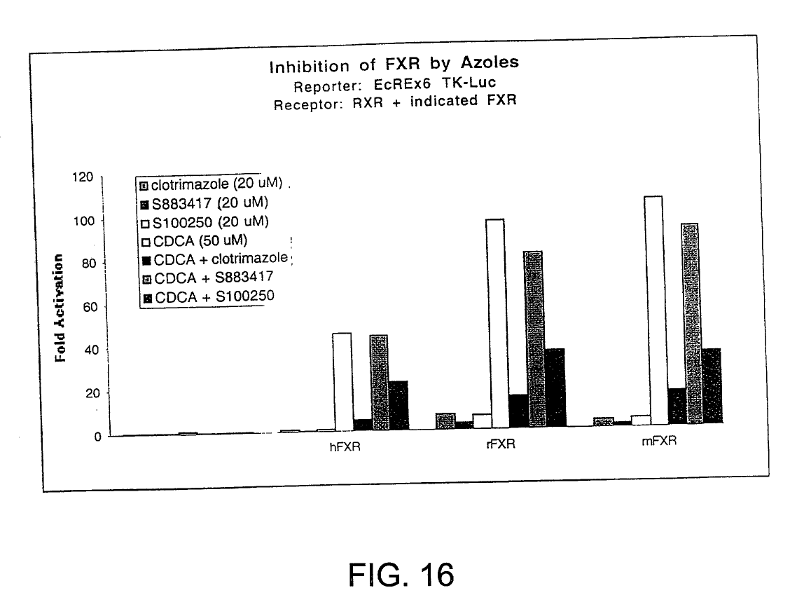

The FXR receptor activation assays disclosed herein may be used not only to test compounds for activation of FXR as above, but also for compounds which antagonize FXR activation. Cells may be treated with known FXR agonists, for example CDCA, or mixtures of FXR agonists in combination with candidate antagonist compounds to determine whether the compounds antagonize FXR activation. Results of such an assay are shown in FIG. 16. See Example 7. The compounds ketonazole, clotrimazole, Compound A and Compound B (see Table II, above) were tested for the ability to antagonize CDCA activation of FXR derived from human, rat and mouse. While ketonazole was inactive, the remaining compounds did demonstrate inhibition of FXR activation. The relative activities of the active compounds was clotrimazole>Compound B>>Compound A. Compound A exhibited significant but weak activity, while clotrimazole was strongly active.

Since farnesoid metabolites had previously been shown to activate FXR (Forman et al., Cell, 81:687-693 (1995)), the activity of one of the most active farnesoid activators, juvenile hormone III (JH III, 50 μM) was tested. This compound was active, but had a far weaker activity relative to the most efficacious bile acids (FIG. 9).

CDCA and CA are both major bile acids produced via the classical pathway, however although CDCA was an extremely effective activator of FXR, CA was inactive. Both CA and conjugated bile acids are relatively hydrophilic compounds that do not readily cross cell membranes. It was possible that no activation of the bile acid nuclear receptor was detected in the assay not because the compounds themselves were not active, but simply because they could not enter the cells in a high enough concentration. A second assay for bile acid nuclear receptor activation was devised which could effectively test for activation by compounds which cannot cross the cell membrane unassisted.

The liver and ileum express tissue-specific bile acid transport proteins for efficient uptake of these compounds. (Craddock et al., Am. J. Physiol., 274:G157-169 (1998)). Neither of these transporters were expressed in the CV-1/COS cells used above. CV-1 cells therefore were co-transfected with a human liver bile acid transporter (accession L21893). Use of this transporter allows hydrophilic bile acid or bile acid-derived compounds or compounds, which due to their structural similarity to bile acid are transported by the transporter, to be tested for activation of the intracellular bile acid nuclear receptor. Bile acid transporters from renal or ileal tissues may also be used efficaciously. Any suitable non-specific transporter may also be used.

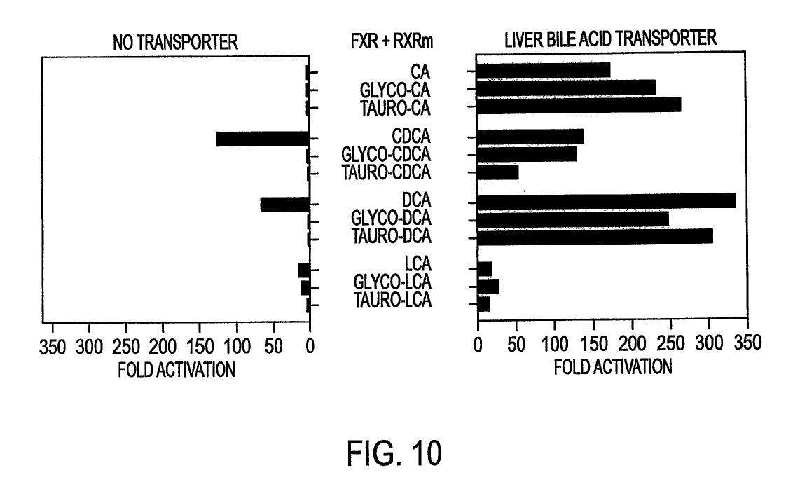

For assay of bile acid nuclear receptor activation by hydrophilic bile acids, CV-1 cells were transfected with an EcRE×6, reporter and expression vectors for β-galactosidase and FXR+RXR alone (FIG. 10, left panel) and additionally with the liver bile acid transporter (FIG. 10, right panel). After transfection, cells were treated with 100 μM concentrations of the indicated bile acid.

Although CA was inactive in the absence of a bile acid transporter (FIG. 10, left panel), coexpression of the liver bile acid transporter allowed CA to exhibit a dramatic 170-fold activation of FXR (FIG. 10, right panel). Similarly, while the glycine and taurine conjugates of CA, CDCA, DCA and LCA were weak or inactive in the first assay, these more hydrophilic bile acids were highly effective in cells expressing the liver bile acid transporter (FIG. 10, compare left and right panels). Similar results were seen with the ileal-specific bile acid transporter (data not shown). Thus, this assay was able to demonstrate that intracellular CA is an effective FXR activator as are the glycine and taurine conjugates of active free bile acids. The assay can be used to test both compounds transported by the bile acid transporter and compounds which are not. The results also demonstrate that FXR and the bile acid transporters share an overlapping specificity.

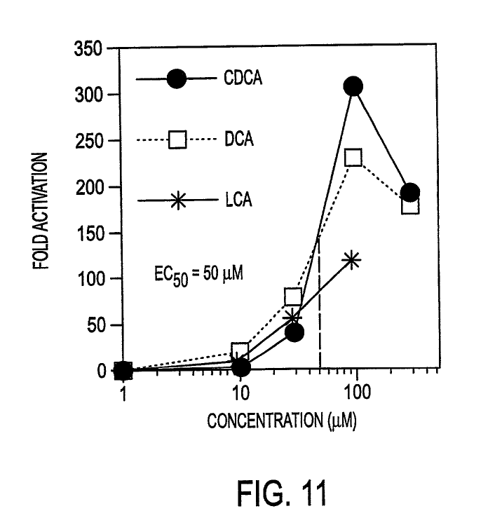

In addition to the structure-activity studies, dose-response analyses were performed for some bile acid nuclear receptor ligands. For these analyses, shown in FIG. 11, CV-1 cells were transfected as above, with the liver bile acid transporter, and treated with the indicated varying concentrations of each bile acid. CDCA, DCA and LCA each displayed an EC₅₀ of approximately 50 μM. The structure-activity relationship (FIGS. 9, 10 and 12) and dose-response profile (FIG. 11) of bile acids for FXR are similar to that reported for the endogenous bile acid sensor (Chiang, Front. Biosci, 3:D176-193 (1998); Kanda et al., Biochem. J., 330:261-265 (1998); Twisk et al., Biochem. J., 305:505-511; Zhang et al., J. Biol. Chem., 273:2424-2428 (1998)). This validates the model used in these studies, showing that the results determined by this assay correlate well with in vivo results.

In addition, the EC₅₀ of bile acids for the bile acid nuclear receptor and the physiologic concentration of the bile acids are closely correlated. For example, the transcriptional effects of CDCA and DCA occur at concentrations of about 50-250 μM (Kanda et al., Biochem. J., 330:261-265 (1998); Twisk et al., Biochem. J., 305:505-511 (1995); Zhang et al., J. Biol. Chem., 273:2424-2428 (1998)). This concentration is very close to the EC₅₀ discovered here for the bile acid nuclear receptor (50 μM) and matches the endogenous concentration of these compounds in bile (CDCA: 10-150 μM; DCA 5 μM) (Matoba et al., J. Lipid Res., 27:1154-1162 (1986)) and intestinal fluid (CDCA: 50 μM; DCA: 320 μM; LCA: 120 μM) (McJunkin et al., Gastroenterol., 80:1454-1464 (1981)). The EC₅₀ for the bile acid nuclear receptor also matches the reported Michaelis constant (Kₘ) of 3-100 μM for liver and ileal bile acid transport proteins (Boyer et al., Am. J. Physiol., 266:G382-G387 (1994); Wong et al., J. Biol. Chem., 269:1340-1347 (1994)). Indeed, the bile acid nuclear receptor responds effectively to bile acids at intracellular concentrations established by the bile acid transporters. See FIG. 10, right panel.

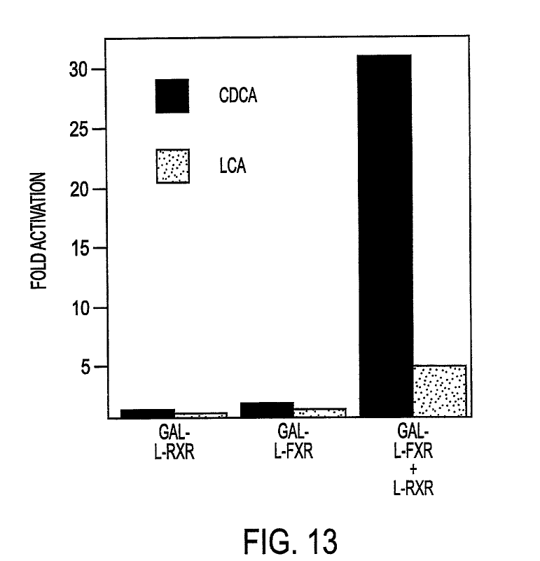

As discussed above, classical nuclear receptors contain modular LBDs that confer ligand-responsiveness to heterologous DNA binding domains. To test whether the bile acid nuclear receptor also requires an interaction with a heterodimeric partner for high affinity binding of the endogenous ligand, the ability of a CDCA and LCA to activate the receptor was tested in cells co-expressing GAL-L-RXR, GAL-L-FXR or GAL-L-FXR plus L-RXR. As expected, CDCA and LCA did not activate the GAL-L-RXR chimera. See FIG. 13. Co-expression of GAL-L-FXR along with the RXR LBD (L-RXR), however, resulted in a complex that was responsive to both CDCA and LCA (FIG. 13). Cells expressing only one LBD (either RXR or FXR) were not activated by either bile acid. These data make it clear that not only is bile acid responsiveness mediated by the FXR LBD, requiring an intact FXR AF2 transactivation domain, but activation of the receptor requires an association with its dimerization partner. In addition, time course experiments indicated that LCA and CDCA activate FXR with the kinetics expected for nuclear receptor ligands, i.e., activity is observed within 1 hour of addition to cells (data not shown).

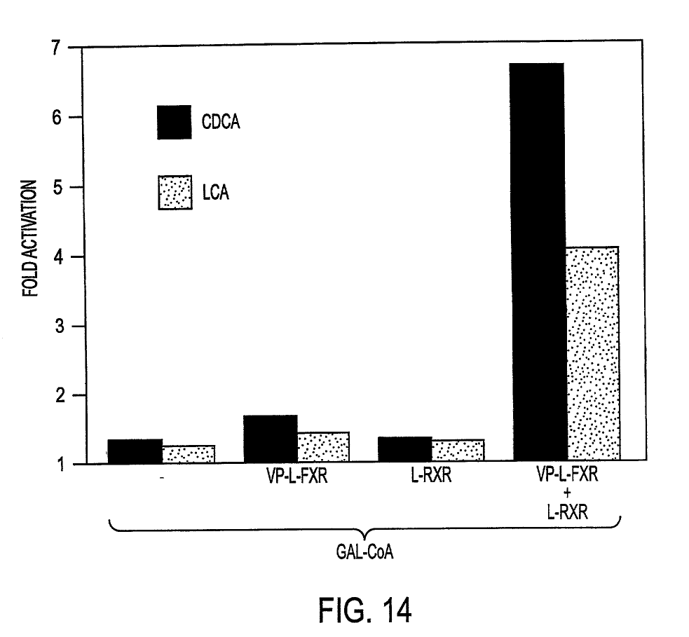

To assess the ability of CDCA and LCA to induce coactivator recruitment, CV-1 cells were transfected with a UAS_{G}×4 reporter and expression vectors for β-galactosidase and GAL-COA (a GAL4 fusion construct containing the 3 receptor interaction domains of the coactivator SRC-1). Where indicated in FIG. 14, cells also were cotransfected with constructs containing the ligand binding domain of RXR (L-RXR) and/or the VP16 transactivation domain fused to the ligand binding domain of FXR (VP-L-FXR). After transfection, cells were treated with 100 μM CDCA or LCA. Neither CDCA nor LCA were able to promote a functional interaction between a GAL4-coactivator fusion protein (GAL-CoA) and a chimera containing the VP16 transactivation domain fused to the FXR LBD (VP-L-FXR). However, in the presence of RXR LBD, the bile acids induced a 4-7 fold increase in activity (FIG. 14).

Coactivator recruitment assays have become established as a reliable method to identify and test the activity of orphan receptor ligands (Blumberg et al., Genes Dev., 12:1269-1277 (1998); Forman et al., Nature, 395:612-615 (1998); Kliewer et al., Cell, 92:73-82 (1998); Krey et al., Mol. Endocrinol., 11:779-791 (1997). In accordance with the present invention, a mammalian two-hybrid in vitro coactivator recruitment assay was developed to examine whether putative ligands could promote a functional association between FXR and a coactivator (or between FXR-RXR heterodimer and coactivator) as a test of a ligand's ability to modify the transcription of genes regulated by the bile acid nuclear receptor. A coactivator is defined as any peptide or polypeptide, whether natural or synthetic, or an active fragment thereof, that functionally interacts with FXR, the FXR-RXR heterodimer or the FXR-RXRm heterodimer in a ligand-dependent manner.

In vitro coactivator recruitment assays were performed by adding the ligand to a mixture of the following components: FXR, 9-cis retinoic acid receptor, a coactivator, and a labeled FXR response element (probe). A polyamino acid containing the receptor interaction domains of co-activator GRIP1 may be used as the coactivator, however any functional coactivator or coactivator complex is contemplated for use in this assay. GRIP1 was expressed in bacteria and purified for these assays. The GST-GRIP1 construct, containing the three receptor interaction domains of mouse GRIP1 (Arg 625-Lys 765, accession U39060) fused to glutathione-S-transferase, was created for bacterial expression of the GRIP1 coactivator. Other suitable coactivators are known in the art, for example PBD/DRIP 205/TRAP 220, and may be used with the inventive methods disclosed here. Response elements suitable for use in this assay may be any nucleic acid probe which is substantially homologous to the target DNA sequence of the bile acid nuclear receptor.

Any response element compatible with the assay system may be used. Oligonucleotide sequences which are substantially homologous to the DNA binding region to which the nuclear receptor binds are contemplated for use with the inventive methods. Substantially homologous sequences (probes) are sequences which bind the ligand activated receptor under the conditions of the assay. Response elements can be modified by methods known in the art to increase or decrease the binding of the response element to the nuclear receptor.

The following response elements were used in the specific assays exemplified here: hsp27 EcRE×6 (Yao et al., Nature, 366:476-479 (1993)), UAS_{G}×4, PPRE×3 (Forman et al., Cell, 81:687-693(1995)), βRE2×3 (Forman et al., Nature, 395:612-615 (1998)), LXRE×3 (Willy et al., Genes Dev., 9:1033-1045 (1995)), T₃RE (MLV)×3 (Perlmann et al., Genes Dev., 7:1411-1422 (1993)), SF1×4 (5′-AGCTTAGCCAAGGTCAGAGAAGCTT; SEQ ID No: 3) and DR0×2 (5′-AAGCTTCAGGTCAAGGTCAGAGAGCTT; SEQ ID No: 4).

After addition of the putative ligand to the mixture of components describe above and mixing, the mixture is incubated under conditions. The formation of complexes in the mixture was analyzed by electrophoretic mobility shift, as shown in FIG. 15, however, any method of separating the complexes formed in the mixture from the individual components may be used, so long as it is sufficient to resolve the labeled complexes from the other components in the mixture. Techniques such as, for example, thin layer chromatography, high pressure liquid chromatography, size exclusion chromatography, sedimentation, immunoseparation techniques, or any other convenient method known in the art may be used.

As expected, FXR-RXR heterodimers failed to recruit coactivator in the absence of ligand (FIG. 15, lane 1). Important in the data provided in FIG. 15, the addition of LCA shifted the majority of the heterodimer into a complex with the coactivator GRIP1 (lane 2). Similar results were seen with glyco-LCA (lane 3) and with LG268 (lane 4). To distinguish between binding through the FXR and RXR subunits, the coactivator recruitment assays were repeated substituting RXRm for RXR. See FIG. 15, lanes 5-8. Significantly, both LCA (lane 6) and glyco-LCA (lane 7) recruited coactivator, while LG268 was inactive (lane 8). These in vitro results demonstrate that LCA and its glycine conjugate are FXR-specific ligands.

While LCA and glyco-LCA were active in the in vitro coactivator recruitment assay, a standard probe of ligand binding activity, other active bile acids including CA, CDCA and DCA were less effective in recruiting GRIP1 or the related coactivators SRC-1 and ACTR (data not shown). Based on their shared structures, activities and activation kinetics, CA, CDCA and DCA are all FXR ligands, though they may also utilize one of the many other nuclear receptor coactivators that have been recently described. See, for example, Blanco et al., Genes Dev., 12:1638-1651 (1998); Fondell et al., Proc. Natl. Acad. Sci. USA, 96:1959-1964 (1999).

The coactivator recruitment assay efficiently detected compounds which were able to form a functional binding relationship with the response element of DNA which regulates a bile acid nuclear receptor (FXR) target gene. Bile acids can inhibit transcription of several genes, including Cyp7a and sterol 27-hydroxylase. (Chiang, Front. Biosci., 3:D176-193 (1998)). In addition to being inhibited by its bile acid end-products, Cyp7a transcription is stimulated by the accumulation of its substrate, cholesterol. This response to cholesterol is mediated by the oxysterol receptor, LXRα (Peet et al., Cell, 93:693-704 (1998)). Thus, control of cholesterol catabolism to bile acids and negative feedback by bile acids, as illustrated by the following diagram:

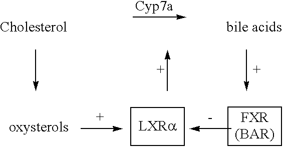

Transcription of an FXR target gene such as Cyp7a, Cyp8b, phospholipid transfer protein, ileal bile acid binding protein, sodium taurocholate cotransporter protein, liver fatty acid binding protein, bile salt export pump or any FXR target gene can be modulated by administering an FXR ligand (agonist or antagonist), an inhibitor of FXR activity) or a ribozyme or antisense therapy directed at the FXR receptor to a cell which expresses the FXR receptor. Exogenous FXR ligands may be used to modify the regulation of Cyp7a (or any other FXR target gene) transcription or the transcription of any gene regulated by FXR. Manipulation of FXR with receptor-binding agonists or antagonists or with any ligand or receptor modulator thus provides a treatment for hypercholesterolemia, elevated triglyceride levels and any other metabolic process disorder which may be controlled through FXR. Of the two major effects of FXR-modulating ligands, agonists of FXR result in lowering of triglyceride levels while antagonists result in lowering of cholesterol levels. Such ligands may be derivatives of natural bile acids, synthetic or semi-synthetic molecules. For example, clotrimazole, Compound A and Compound B all have been found to antagonize the actions of the natural agonist, CDCA with varying potency. Compounds which bind to and which are useful for modulating FXR include compounds of Formula I:

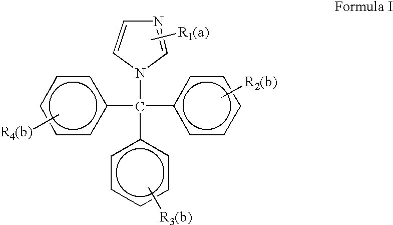

wherein (a) represents an integer from 0 to 3 and each (b) may be the same or different and represents an integer from 0 to 5;

wherein each R₁, R₂, R₃ and R₄ may be the same as or different from any other R₁, R₂, R₃ or R₄ and represents a moiety selected from the group consisting of C₁₋₄ alkyl, C₁₋₄ alkenyl, aryl, alkylaryl, halo, trihalomethyl, furanyl, thiophenyl, pyrrolyl, pyrazolyl, diazolyl, triazolyl, tetrazolyl, dithiolyl, oxathiolyl, oxazolyl, isoxazolyl, thiazolyl, isothiazolyl, oxadiazolyl, oxatriazolyl, dioxazolyl, isoxazinyl and piperazinyl, and

wherein said moiety may be unsubstituted or substituted with one or more substituent selected from the group consisting from methyl, ethyl, amino, halo, trihalomethyl and nitro. These compounds also are useful as lead compounds for the discovery of new compounds in screening assays. The assay may be of any of those described herein or any other assay known in the art.

The method of the invention may be used for screening putative FXR ligands which may act as agonists or antagonists in the FXR receptor or putative FXR blockers and therefore may be used therapeutically or prophylactically for the control of cholesterol metabolism. The invention also provides compounds produced by such methods.

The assays described above and exemplified below provide methods of selecting compounds which modulate the transcription of genes regulated by FXR. For example, compounds according to Formula I described above are suitable compounds for use in the methods of this invention. In particular, clotrimazole, Compound A and Compound B were selected by the inventive method and have been found to modulate the transcription of endogenous genes or reporter genes controlled by FXR. The invention is further described and illustrated in the following examples, which are not intended to be limiting.

### EXAMPLES

### Example 1

### Transient Transfection Assay for FXR Activity

CV-1 cells were grown in Dulbecco's Modified Eagle's medium supplemented with 10% resin-charcoal stripped fetal bovine serum, 50 U/ml penicillin G and 50 μg/ml streptomycin sulfate (DMEM-FBS) at 37° C. in 5% CO₂. One day prior to transfection, cells were plated to 50-80% confluence using phenol-red free DMEM-FBS. Cells were transiently transfected by lipofection as described (Forman et al., Cell, 81:687-693 (1995). Luciferase reporter constructs (300 ng/10⁵ cells) containing the herpes virus thymidine kinase promoter (−105/+51) linked to six copies of the ecdysone response element (EcRE×6) and cytomegalovirus driven expression vectors (20-50 ng/10⁵ cells) were added, along with CMV-β-gal as an internal control. Mammalian expression vectors were derived from a CMV expression vector which contains the cytomegalovirus promoter/enhancer followed by a bacteriophage T7 promoter for transcription in vitro. Two assays were performed in parallel, one using cells transfected with an EcRE×6 reporter and expression vectors containing β-gal, FXR and RXR, and one using cells transfected with an EcRE×6 reporter and expression vectors containing β-gal, FXR and RXRm. After incubation with liposomes for 2 hours, the liposomes were removed and cells treated for approximately 45 hours with phenol-red free DMEM-FBS containing 100 μM CDCA. After exposure to ligand, the cells were harvested and assayed for luciferase and β-galactosidase activity according to known methods. See FIG. 9. All points were assayed in triplicate and varied by less than 15%. Each experiment was repeated three or more times with similar results.

### Example 2

### Transient Transfection Assay for FXR Activity Suitable for Hydrophilic Compounds

An assay was performed in Example 1 using cells transfected with an EcRE×6 reporter and expression vectors containing β-gal, FXR and RXR or RXRm, with the exception that the cells were also co-transfected with a pcDNA expression vector for the human liver bile acid transporter. See FIG. 10.

### Example 3

### Screening Assay for Compounds which Modulate the Transcription of a Bile Acid Nuclear Receptor Target Gene

CV-1 cells are grown in Dulbecco's Modified Eagle's medium (DMEM) supplemented with 10% resin-charcoal stripped fetal bovine serum. 50 U/ml penicillin G and 50 μg/ml streptomycin sulfate at 37° C. in 5% CO₂. Cells are plated to 50-80% confluence one day prior to transfection using phenol red-free DMEM-FBS. The cells are transfected by lipofection using N-[1-(2,3-dioleoyloxy)propyl]-N,N,N-ammonium methyl sulfate according to the instructions of the manufacturer (Boehringer Mannheim).

The CV-1 cells are transfected with expression vectors containing FXR and/or RXR and a luciferase reporter construct containing the herpesvirus thymidine kinase promoter (−105/+51) linked to the indicated number of copies of the response element hsp27 EcRE×6. A parallel assay is performed in which the ligand-binding domain of RXR accession no. X52773) is replaced with the ligand-binding domain RXRm. After transfection, cells are treated with varying concentrations of candidate bile acid receptor agonist or antagonist compounds for approximately 45 hours in phenol red free DMEM-FBS. After exposure to the compounds, cells are harvested and assayed for luciferase and β-galactosidase activity.

### Example 4

### Screening Assay for Compounds which Modulate the Transcription of a Bile Acid Nuclear Receptor Target Gene

A screening assay is performed according to Example 3 with the exception that the cells are also co-transfected with a pcDNA expression vector for the human liver bile acid transporter.

### Example 5

### Screening Assay for Compounds which Modulate the Transcription of a Bile Acid Nuclear Receptor Target Gene

A screening assay is performed according to Example 3 with the exception that the parallel assay using the expression construct containing RXRm is omitted.

### Example 6

### Coactivator Recruitment Assay

GST-GRIP1 was expressed in E. coli and purified on glutathione-sepharose columns. In vitro translated FXR+RXR (FIG. 15, left panel) or FXR+RXRm (FIG. 15, right panel) and GST-GRIP1 (5 μg) were incubated for 30 minutes at room temperature with 100,000 cpm of a Klenow-labeled hsp27 EcRE probe (5′-AGCTCGATGGACAAGTGCATTGAACCCTTGAAGCTT; SEQ ID NO: 5) in 10 mM Tris pH 8, 50 mM KCl, 6% glycerol, 0.05% NP-40, 1 mM DTT, 12.5 ng/μl poly dI-dC and the ligands to be tested for coactivator recruitment. Complexes were electrophoresed through a 5% polyacrylamide gel in 0.5×TBE (45 mM Tris base, 45 mM boric acid, 1 mM EDTA) at room temperature. Electrophoretic mobility indicated recruitment of coactivator.

### Example 7

### Selection of FXR Receptor Antagonist Compounds

CV-1 cells were grown in DMEM supplemented with 10% resin-charcoal stripped fetal bovine serum, 50 U/ml penicillin G and 50 μg/ml streptomycin sulfate at 37° C. in 5% CO₂. Cells were plated to 50-80% confluence one day prior to transfection using phenol red-free DMEM-FBS. The cells were transfected by lipofection using N-[1-(2,3-dioleoyloxy)propyl]-N,N,N-ammonium methyl sulfate according to manufacture's instructions (Boehringer Mannheim). The cells were transfected with a CMV expression vector containing the FXR indicated in FIG. 16 (none, −; human, hFXR; rat, rFXR; or mouse, mFXR) and RXR. The luciferase reporter construct contained the herpesvirus thymidine kinase promoter (−105/+51) linked to six copies of the response element hsp27 EcRE. After transfection, the cells were treated with 50 μM CDCA and 20 μM of a synthetic azole compound or both in phenol red-free DMEM-FBS. The chemical structures of the azole compounds used (clotrimazole, Compound A and Compound B) are provided in Table II, above. After exposure to the agonist and candidate antagonist compounds, cells were harvested and assayed for luciferase and β-galactosidase activity. Results are provided in FIG. 16. All three of these azole compounds displayed antagonist properties, with clotrimazole having the strongest effect. Therefore, clotrimazole, Compound A and Compound B were selected from the screen as FXR receptor antagonists.

1. A method of modulating an FXR-dependent physiological process which comprises modulating the activation of FXR.

2. A method of claim 1 wherein said FXR-dependent physiological process is cholesterol metabolism.

3. A method of claim 1 which comprises modulating expression of an FXR target gene.

4. A method of claim 3 wherein said FXR target gene encodes a protein or peptide selected from the group consisting of cholesterol 7a-hydroxylase, Cyp8b, phospholipid transfer protein, ileal bile acid binding protein, sodium taurocholate cotransporter protein, liver fatty acid binding protein and bile salt export pump.

5. A method of claim 4 wherein said FXR target gene encodes Cyp7a.

6. A method of claim 5 wherein Cyp7a expression is modulated by modulating the activation of FXR.

7. A method of claim 2, wherein cholesterol catabolism is increased by upregulating expression of the gene encoding Cyp7a to a level of expression that is substantially more than that which occurs naturally in said cell.

8. A method of claim 7, wherein upregulation of expression of the gene encoding Cyp7a is achieved by inhibiting activation of FXR.

9. A method of claim 8, wherein upregulation of expression of the gene encoding Cyp7a is achieved by contacting said FXR with an FXR antagonist.

10. A method of claim 2, wherein cholesterol metabolism is decreased by downregulating expression of the gene encoding Cyp7a to a level that is substantially less than that which occurs naturally in said cell.

11. A method of claim 10, wherein downregulation of expression of the gene encoding Cyp7a is achieved by increasing activation of FXR.

12. A method of claim 11, wherein downregulation of expression of the gene encoding Cyp7a is achieved by contacting said FXR with an FXR agonist.

13. A method of claim 4, wherein the expression of Cyp8b is upregulated to a level of expression substantially more than that which occurs naturally in said cell.

14. A method of claim 4, wherein the expression of Cyp8b is downregulated to a level of expression substantially less than that which occurs naturally in said cell.

15. A method of claim 13, wherein upregulation of expression of the gene encoding Cyp8b is achieved by inhibiting activation of FXR.

16. A method of claim 14, wherein downregulation of expression of the gene encoding Cyp8b is achieved by increasing activation of FXR.

17. A method of claim 4, wherein the expression of phospholipid transfer protein is upregulated to a level of expression substantially more than that which occurs naturally in said cell.

18. A method of claim 4, wherein the expression of phospholipid transfer protein is downregulated to a level of expression substantially less than that which occurs naturally in said cell.

19. A method of claim 17, wherein upregulation of expression of the gene encoding phospholipid transfer protein is achieved by increasing activation of FXR.

20. A method of claim 18, wherein downregulation of expression of the gene encoding phospholipid transfer protein is achieved by inhibiting activation of FXR.

21. A method of claim 4, wherein the expression of ileal bile acid binding protein is upregulated to a level of expression substantially more than that which occurs naturally in said cell.

22. A method of claim 4, wherein the expression of ileal bile acid binding protein is downregulated to a level of expression substantially less than that which occurs naturally in said cell.

23. A method of claim 21, wherein upregulation of expression of the gene encoding ileal bile acid binding protein is achieved by increasing activation of FXR.

24. A method of claim 22, wherein downregulation of expression of the gene encoding ileal bile acid binding protein is achieved by inhibiting activation of FXR.

25. A method of claim 4, wherein the expression of sodium taurocholate cotransporter protein is upregulated to a level of expression substantially more than that which occurs naturally in said cell.

26. A method of claim 4, wherein the expression of sodium taurocholate cotransporter protein is downregulated to a level of expression substantially less than that which occurs naturally in said cell.

27. A method of claim 25, wherein upregulation of expression of the gene encoding sodium taurocholate cotransporter protein is achieved by inhibiting activation of FXR.

28. A method of claim 26, wherein downregulation of expression of the gene encoding sodium taurocholate cotransporter protein is achieved by increasing activation of FXR.

29. A method of claim 4 wherein the expression of liver fatty acid binding protein is upregulated to a level of expression substantially more than that which occurs naturally in said cell.

30. A method of claim 4 wherein the expression of liver fatty acid binding protein is downregulated to a level of expression substantially less than that which occurs naturally in said cell.

31. A method of claim 29 wherein upregulation of expression of the gene encoding liver fatty acid binding protein is achieved by increasing activation of FXR.

32. A method of claim 30 wherein downregulation of expression of the gene encoding liver fatty acid binding protein is achieved by inhibiting activation of FXR.

33. A method of claim 4, wherein the expression of bile salt export pump is upregulated to a level of expression substantially more than that which occurs naturally in said cell.

34. A method of claim 4, wherein the expression of bile salt export pump is downregulated to a level of expression substantially less than that which occurs naturally in said cell.

35. A method of claim 33, wherein upregulation of expression of the gene encoding bile salt export pump is achieved by increasing activation of FXR.

36. A method of claim 34, wherein downregulation of expression of the gene encoding bile salt export pump is achieved by inhibiting activation of FXR.

37. A method of claim 1, wherein said FXR-dependent physiological process is triglyceride metabolism.

38. A method of claim 37, wherein triglyceride levels are decreased by increasing activation of FXR.

39. A method of claim 37, wherein triglyceride levels are increased by inhibiting activation FXR.

40. A method of claim 1, which comprises contacting said FXR with a compound according to Formula I: 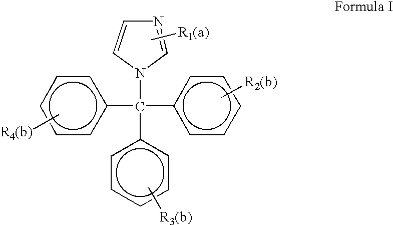
wherein (a) represents an integer from 0 to 3 and each (b) may be the same or different and represents an integer from 0 to 5;
wherein each R₁, R₂, R₃, and R₄ may be the same as or different from any other R₁, R₂, R₃ or R₄ and represents a moiety selected from the group consisting of C₁₋₄ alkyl, C₁₋₄ alkenyl, aryl, alkylaryl, halo, trihalomethyl, furanyl, thiophenyl, pyrrolyl, pyrazolyl, diazolyl, triazolyl, tetrazolyl, dithiolyl, oxathiolyl, oxazolyl, isoxazolyl, thiazolyl, isothiazolyl, oxadiazolyl, oxatriazolyl, dioxazolyl, isoxazinyl and piperazinyl, and
wherein said moiety may be unsubstituted or substituted with one or more substituent selected from the group consisting from methyl, ethyl, amino, halo, trihalomethyl and nitro.

41. A method of claim 40, wherein said compound of Formula I is selected from the group consisting of clotrimazole, Compound A and Compound B.

42. A method of claim 40, wherein said compound of Formula I is clotrimazole.

43. A method of claim 40, wherein said compound of Formula I is Compound B.

44. A method for screening for pharmacologically active compounds which comprises determining whether a compound activates or inhibits activation of the FXR receptor.

45. A method of claim 44, which is a method for screening for compounds capable of modulating an FXR-dependent physiological process selected from the group consisting of cholesterol catabolism and triglyceride metabolism.

46. A method of claim 45, in which the method comprises determining whether the compound inhibits FXR activation, thereby increasing cholesterol catabolism.

47. A method of screening for compounds useful in modulating FXR-mediated gene transcription which comprises contacting a mixture of FXR and RXR with a compound and determining whether said compound promotes interaction between FXR-RXR heterodimer and coactivator or between FXR and coactivator.

48. A method of screening compounds for FXR antagonist activity which comprises contacting a mixture of FXR and RXR and a known FXR agonist with at least one of said compounds and determining whether said compound inhibits the agonist-promoted activation of FXR or of an FXR-RXR heterodimer.

49. A method of claim 48, wherein said known FXR agonist is a compound of Formula I: 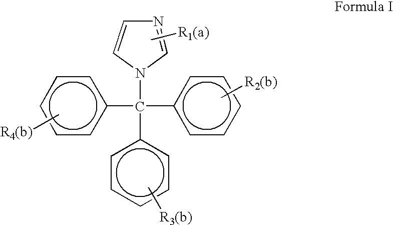
wherein (a) represents an integer from 0 to 3 and each (b) may be the same or different and represents an integer from 0 to 5;
wherein each R₁, R₂, R₃, and R₄, may be the same as or different from any other R₁, R₂, R₃ or R₄ and represents a moiety selected from the group consisting of C₁₋₄ alkyl, C₁₋₄ alkenyl, aryl, alkylaryl, halo, trihalomethyl, furanyl, thiophenyl, pyrrolyl, pyrazolyl, diazolyl, triazolyl, tetrazolyl, dithiolyl, oxathiolyl, oxazolyl, isoxazolyl, thiazolyl, isothiazolyl, oxadiazolyl, oxatriazolyl, dioxazolyl, isoxazinyl and piperazinyl, and
wherein said moiety may be unsubstituted or substituted with one or more substituent selected from the group consisting from methyl, ethyl, amino, halo, trihalomethyl and nitro.

50. A method of claim 49, wherein said compound of Formula I is selected from the group consisting of clotrimazole, Compound A and Compound B.

51. A method of claim 50, wherein said compound of Formula I is clotrimazole.

52. A method of claim 50, wherein said compound of Formula I is Compound B.

53. A method of claim 47, 48, 49, or 50, wherein the RXR is an RXR mutant (RXRm) which contains a functional DNA-binding domain and which has a mutation in the ligand-binding domain which prevents substantial activation by RXR ligands but which does not otherwise substantially affect the ability of the RXR mutant receptor to form heterodimers with FXR.

54. A method for screening compounds for cholesterol catabolism-modulating activity which comprises:
(1) providing a first mixture which contains
(i) an FXR receptor,
(ii) an RXR receptor, and
(iii) a labeled DNA probe which contains a sequence of nucleotides to which the DNA-binding domain of a ligand-FXR-RXR complex specifically binds;
(2) providing a second mixture which contains
(i) an FXR receptor,
(ii) an RXR mutant receptor (“RXRm”) which contains a functional DNA-binding domain and which has a mutation in the ligand-binding domain which prevents substantial activation by RXR ligands but which does not otherwise substantially affect the ability of the RXR mutant receptor to form heterodimers with FXR or of such heterodimers to recruit coactivator, and
(iii) a labeled DNA probe which contains a sequence of nucleotides to which the DNA-binding domain of a ligand-FXR-RXR complex specifically binds;
(3) contacting said first and second mixtures with the compound being screened;
(4) determining whether the compound causes interaction of an FXR-RXR heterodimer with coactivator or of FXR with coactivator; and
(5) determining whether the compound being screened causes interaction of an FXR-RXRm heterodimer with coactivator.

55. A method of claim 54, which further comprises contacting said first and second mixtures with a known FXR ligand and selecting compounds that inhibit the ability of said known FXR ligand to cause the FXR-RXR heterodimer or FXR to interact with coactivator.

56. A method of claim 55, wherein said known FXR agonist is a compound of Formula I: 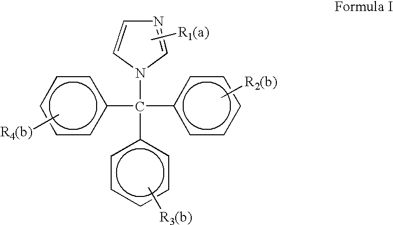
wherein (a) represents an integer from 0 to 3 and each (b) may be the same or different and represents an integer from 0 to 5;
wherein each R₁, R₂, R₃, and R₄, may be the same as or different from any other R₁, R₂, R₃ or R₄ and represents a moiety selected from the group consisting of C₁₋₄ alkyl, C₁₋₄ alkenyl, aryl, alkylaryl, halo, trihalomethyl, furanyl, thiophenyl, pyrrolyl, pyrazolyl, diazolyl, triazolyl, tetrazolyl, dithiolyl, oxathiolyl, oxazolyl, isoxazolyl, thiazolyl, isothiazolyl, oxadiazolyl, oxatriazolyl, dioxazolyl, isoxazinyl and piperazinyl, and
wherein said moiety may be unsubstituted or substituted with one or more substituent selected from the group consisting from methyl, ethyl, amino, halo, trihalomethyl and nitro.

57. A method of claim 56, wherein said compound of Formula I is selected from the group consisting of clotrimazole, Compound A and Compound B.

58. A method of claim 48, wherein said compound of Formula I is clotrimazole.

59. A method of claim 48, wherein said compound of Formula I is Compound B.

60. A method of claim 55, which further comprises selecting compounds that do not cause substantial interaction of FXR, the FXR-RXR heterodimer or the FXR-RXRm heterodimer with coactivator.

61. A method of screening for compounds useful in modulating FXR-mediated gene transcription which comprises contacting a mixture of FXR, RXR and a coactivator with a compound and determining whether said compound promotes coactivator recruitment by an FXR-RXR heterodimer or FXR.

62. A method of screening compounds for FXR antagonist activity which comprises contacting a mixture of FXR, RXR, a coactivator and a known FXR agonist with at least one of said compounds and determining whether said compound inhibits the agonist-promoted coactivator recruitment by an FXR-RXR heterodimer or FXR.

63. A method of claim 62, wherein said known FXR agonist is a compound of Formula I: 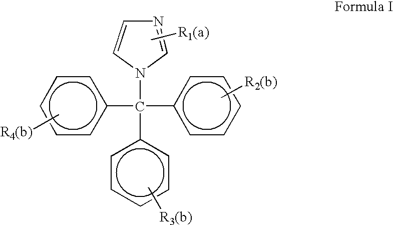
wherein (a) represents an integer from 0 to 3 and each (b) may be the same or different and represents an integer from 0 to 5;
wherein each R₁, R₂, R₃, and R₄, may be the same as or different from other R₁, R₂, R₃, or R₄ and represents a moiety selected from the group consisting of C₁₋₄ alkyl, C₁₋₄ alkenyl, aryl, alkylaryl, halo, trihalomethyl, furanyl, thiophenyl, pyrrolyl, pyrazolyl, diazolyl, triazolyl, tetrazolyl, dithiolyl, oxathiolyl, oxazolyl, isoxazolyl, thiazolyl, isothiazolyl, oxadiazolyl, oxatriazolyl, dioxazolyl, isoxazinyl and piperazinyl, and
wherein said moiety may be unsubstituted or substituted with one or more substituent selected from the group consisting from methyl, ethyl, amino, halo, trihalomethyl and nitro.

64. A method of claim 63, wherein said compound of Formula I is selected from the group consisting of clotrimazole, Compound A and Compound B.

65. A method of claim 64, wherein said compound of Formula I is clotrimazole.

66. A method of claim 64, wherein said compound of Formula I is Compound B.

67. A method of claim 61 or 62, wherein the RXR is an RXR mutant (“RXRm”) which contains a functional DNA-binding domain and which has a mutation in the ligand-binding domain which prevents substantial activation by RXR ligands but which does not otherwise substantially affect the ability of the RXR mutant receptor to form heterodimers with FXR or of such heterodimers to recruit coactivator.

68. A method of claim 53, in which the RXRm is an Asp₃₃₂→Pro mutant of human RXRα.

69. A method of claim 54, 55 or 60, in which the RxRm is an Asp₃₂₂→Pro mutant of human RXRα.

70. A method of claim 67, in which the RXRm is an Asp₃₂₂→Pro mutant of human RXRα.

71. A method of claim 61 or 62, in which the coactivator is a polypeptide or active fragment thereof which contains a peptide motif that interacts with the FXR-RXR heterodimer in a ligand-dependant manner.

72. A method of claim 67, in which the coactivator is a polypeptide or active fragment thereof which contains a peptide motif that interacts with the FXR-RXR heterodimer in a ligand-dependent manner.

73. A method of claim 61 or 62, in which the coactivator is selected from SRC-1, GRIP, ACTR and PBP/DRIP205/TRAP220.

74. A method of claim 67, in which the coactivator is selected from SRC-1, GRIP, ACTR and PBP/DRIP205/TRAP220.

75. A method of claims 61 or 62, in which the coactivator is GRIP1.

76. A method of claim 67, in which the coactivator is GRIP1.

77. A method of claim 54, 55, or 60 in which the DNA probe comprises a nucleotide sequence selected from the group consisting of SEQ ID NO: 3 and SEQ ID NO: 4.

78. A method of screening for compounds useful in modulating FXR-mediated gene transcription, which comprises:
(a) transfecting mammalian cells with a gene encoding FXR under control of an operative promoter;
(b) transfecting said cells with an operative reporter gene under control of a promoter linked to a DNA sequence which encodes an operative response element to which ligand-activated FXR or FXR complex binds to initiate transcription of said reporter gene;
(c) culturing said cells in the presence of a compound being screened; and
(d) monitoring said cells for transcription or expression of the reporter gene as an indication of FXR activation.

79. A method of claim 78, wherein said cells are transfected with a gene encoding RXR or RXRm under control of an operative promoter.

80. A method of claim 78, wherein said cells are transfected with a gene encoding a bile acid transporter molecule under control of an operative promoter.

81. A method of claim 80, wherein said cells are transfected with a gene encoding a bile acid transporter molecule under control of an operative promoter.

82. A method of claim 78, 79, 80 or 81 wherein said cells are cultured in the presence of a known FXR ligand and the diminution of transcription or expression of the reporter gene is an indication that the compound being screened is an FXR antagonist.

83. A method of claim 81, wherein said known FXR agonist is a compound of Formula I: 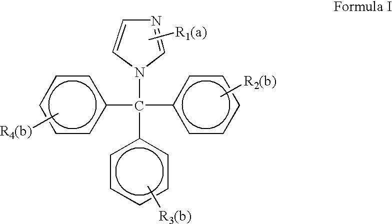
wherein (a) represents an integer from 0 to 3 and each (b) may be the same or different and represents an integer from 0 to 5;
wherein each R₁, R₂, R₃, and R₄, may be the same as or different from any other R₁, R₂, R₃, or R₄ and represents a moiety selected from the group consisting of C₁₋₄ alkyl, C₁₋₄ alkenyl, aryl, alkylaryl, halo, trihalomethyl, furanyl, thiophenyl, pyrrolyl, pyrazolyl, diazolyl, triazolyl, tetrazolyl, dithiolyl, oxathiolyl, oxazolyl, isoxazolyl, thiazolyl, isothiazolyl, oxadiazolyl, oxatriazolyl, dioxazolyl, isoxazinyl and piperazinyl, and
wherein said moiety may be unsubstituted or substituted with one or more substituent selected from the group consisting from methyl, ethyl, amino, halo, trihalomethyl and nitro.

84. A method of claim 83, wherein said compound of Formula I is selected from the group consisting of clotrimazole, Compound A and Compound B.

85. A method of claim 83, wherein said compound of Formula I is clotrimazole.

86. A method of claim 83, wherein said compound of Formula I is Compound B.

87. A method of claim 77, in which the method is used to identify compounds that are useful for increasing cholesterol catabolism.

88. A non-naturally occurring compound selected by the method of claims 47, 48, 49, 50, 54, 55, 56, 57, 60, 61, 62, 78, 79, 80 or 81.

89. A non-naturally occurring compound selected by the method of claim 53.

90. A non-naturally occurring compound selected by a method of claim 63.

91. A pharmaceutical composition comprising a therapeutically or prophylactically effective amount of a compound of claim 88 in combination with a pharmaceutically acceptable carrier.

92. A pharmaceutical composition comprising a therapeutically or prophylactically effective amount of a compound of claim 89 in combination with a pharmaceutically acceptable carrier.

93. A pharmaceutical composition comprising a therapeutically or prophylactically effective amount of a compound of claim 90 in combination with a pharmaceutically acceptable carrier.

94. A method of treating a mammal for hypercholesterolemia which comprises administering an effective amount of the pharmaceutical composition of claim 91.

95. A method of treating a mammal for hypercholesterolemia which comprises administering an effective amount of the pharmaceutical composition of claim 92.

96. A method of treating a mammal for hypercholesterolemia which comprises administering an effective amount of the pharmaceutical composition of claim 93.


SEQUENCE LISTING (5 sequences)

SEQ ID NO: 1 - protein, 9 aa, Simian virus 40
APKKKRKVG

SEQ ID NO: 2 - DNA, 27 bp, Artificial sequence
  DR1 probe
agctaccaggtcaaaggtcacgtagct

SEQ ID NO: 3 - DNA, 25 bp, Artificial sequence
  SF1 x 4 Response Element
agcttagccaaggtcagagaagctt

SEQ ID NO: 4 - DNA, 27 bp, Artificial sequence
  DR0 x 2 Response Element
aagcttcaggtcaaggtcagagagctt

SEQ ID NO: 5 - DNA, 36 bp, Artificial sequence
  hsp27 EcRE probe
agctcgatggacaagtgcattgaacccttgaagctt